SEQUENCE LISTING (76 sequences)

SEQ ID NO: 1 - DNA, 17 bp, Homo sapiens
  source 1..17: mol_type = other DNA
cgagccgcctggatacc

SEQ ID NO: 2 - DNA, 25 bp, Homo sapiens
  source 1..25: mol_type = other DNA
cagttccgaaaaccaacaaaataga

SEQ ID NO: 3 - DNA, 24 bp, Homo sapiens
  source 1..24: mol_type = other DNA
attattttggcacaacaggaagct

SEQ ID NO: 4 - DNA, 22 bp, Homo sapiens
  source 1..22: mol_type = other DNA
tccgcataggactgaccaagat

SEQ ID NO: 5 - DNA, 20 bp, Homo sapiens
  source 1..20: mol_type = other DNA
accgaaatcacagccagtag

SEQ ID NO: 6 - DNA, 22 bp, Homo sapiens
  source 1..22: mol_type = other DNA
cctcctcactcagctcatattc

SEQ ID NO: 7 - DNA, 22 bp, Homo sapiens
  source 1..22: mol_type = other DNA
gattgagtcctggctgttatga

SEQ ID NO: 8 - DNA, 23 bp, Homo sapiens
  source 1..23: mol_type = other DNA
gggtttacactgacctttaggat

SEQ ID NO: 9 - DNA, 20 bp, Homo sapiens
  source 1..20: mol_type = other DNA
cgggctttggacaccaatta

SEQ ID NO: 10 - DNA, 18 bp, Homo sapiens
  source 1..18: mol_type = other DNA
gggcgcacacagcagttc

SEQ ID NO: 11 - DNA, 20 bp, Homo sapiens
  source 1..20: mol_type = other DNA
gccctctgtgccacagatgt

SEQ ID NO: 12 - DNA, 15 bp, Homo sapiens
  source 1..15: mol_type = other DNA
ccccgctgccaccat

SEQ ID NO: 13 - DNA, 25 bp, Homo sapiens
  source 1..25: mol_type = other DNA
ggactcttcggagaactttcagatc

SEQ ID NO: 14 - DNA, 19 bp, Homo sapiens
  source 1..19: mol_type = other DNA
gggcacaggaaaacatcga

SEQ ID NO: 15 - DNA, 23 bp, Homo sapiens
  source 1..23: mol_type = other DNA
catctgatgtctgaagaggagtg

SEQ ID NO: 16 - DNA, 22 bp, Homo sapiens
  source 1..22: mol_type = other DNA
tatgtggttctggctcatgaat

SEQ ID NO: 17 - DNA, 20 bp, Homo sapiens
  source 1..20: mol_type = other DNA
tacatcgggccttgcaaata

SEQ ID NO: 18 - DNA, 21 bp, Homo sapiens
  source 1..21: mol_type = other DNA
tgtcctcatccctctcataca

SEQ ID NO: 19 - DNA, 22 bp, Homo sapiens
  source 1..22: mol_type = other DNA
gcgatcaactgtgagtgatagg

SEQ ID NO: 20 - DNA, 22 bp, Homo sapiens
  source 1..22: mol_type = other DNA
ggttgggttcatcttcggtatc

SEQ ID NO: 21 - DNA, 18 bp, Homo sapiens
  source 1..18: mol_type = other DNA
cctggctgctctcaacat

SEQ ID NO: 22 - DNA, 22 bp, Homo sapiens
  source 1..22: mol_type = other DNA
cggatctccaagtactcaaagt

SEQ ID NO: 23 - DNA, 21 bp, Homo sapiens
  source 1..21: mol_type = other DNA
cctgccatcctattgtgagtt

SEQ ID NO: 24 - DNA, 21 bp, Homo sapiens
  source 1..21: mol_type = other DNA
agcttcatagcactcaatccc

SEQ ID NO: 25 - DNA, 22 bp, Homo sapiens
  source 1..22: mol_type = other DNA
gactcaggtacccgatgatcta

SEQ ID NO: 26 - DNA, 21 bp, Homo sapiens
  source 1..21: mol_type = other DNA
gctggctataccttgtgaagt

SEQ ID NO: 27 - DNA, 19 bp, Homo sapiens
  source 1..19: mol_type = other DNA
ggagacttgcctggtgaaa

SEQ ID NO: 28 - DNA, 21 bp, Homo sapiens
  source 1..21: mol_type = other DNA
ctggcttgttcctcactactc

SEQ ID NO: 29 - DNA, 20 bp, Homo sapiens
  source 1..20: mol_type = other DNA
cttggcagccttcctgattt

SEQ ID NO: 30 - DNA, 23 bp, Homo sapiens
  source 1..23: mol_type = other DNA
ttctttagcactccttggcaaaa

SEQ ID NO: 31 - DNA, 21 bp, Homo sapiens
  source 1..21: mol_type = other DNA
ccagggacctctctctaatca

SEQ ID NO: 32 - DNA, 20 bp, Homo sapiens
  source 1..20: mol_type = other DNA
tcagcttgagggtttgctac

SEQ ID NO: 33 - DNA, 23 bp, Homo sapiens
  source 1..23: mol_type = other DNA
gagtagaacatcatgggacagag

SEQ ID NO: 34 - DNA, 21 bp, Homo sapiens
  source 1..21: mol_type = other DNA
acgcgaagagaagcagaatta

SEQ ID NO: 35 - DNA, 21 bp, Homo sapiens
  source 1..21: mol_type = other DNA
atggacaagctgaggaagatg

SEQ ID NO: 36 - DNA, 22 bp, Homo sapiens
  source 1..22: mol_type = other DNA
cccatgtgtcgaagaagatagg

SEQ ID NO: 37 - DNA, 22 bp, Homo sapiens
  source 1..22: mol_type = other DNA
ctgtatcatctggtcacggaag

SEQ ID NO: 38 - DNA, 21 bp, Homo sapiens
  source 1..21: mol_type = other DNA
cgtttggtttgctcctcaatc

SEQ ID NO: 39 - DNA, 20 bp, Homo sapiens
  source 1..20: mol_type = other DNA
gtgccaggctttgtggattt

SEQ ID NO: 40 - DNA, 22 bp, Homo sapiens
  source 1..22: mol_type = other DNA
ggatctctagccaggcacattc

SEQ ID NO: 41 - DNA, 22 bp, Homo sapiens
  source 1..22: mol_type = other DNA
aatggaagggcagcacaactac

SEQ ID NO: 42 - DNA, 23 bp, Homo sapiens
  source 1..23: mol_type = other DNA
tttctgcggattttatcaacgat

SEQ ID NO: 43 - DNA, 21 bp, Homo sapiens
  source 1..21: mol_type = other DNA
aacaaggagctggatgaagag

SEQ ID NO: 44 - DNA, 22 bp, Homo sapiens
  source 1..22: mol_type = other DNA
ctggcatgagtttctgtctagt

SEQ ID NO: 45 - DNA, 19 bp, Homo sapiens
  source 1..19: mol_type = other DNA
ggtggccaaggtggaatac

SEQ ID NO: 46 - DNA, 22 bp, Homo sapiens
  source 1..22: mol_type = other DNA
cactccaaatgctcctctaacc

SEQ ID NO: 47 - DNA, 23 bp, Homo sapiens
  source 1..23: mol_type = other DNA
gtgggtcttctccatctctatct

SEQ ID NO: 48 - DNA, 20 bp, Homo sapiens
  source 1..20: mol_type = other DNA
tagaggatgtggtcggtgtt

SEQ ID NO: 49 - DNA, 20 bp, Homo sapiens
  source 1..20: mol_type = other DNA
cttggcagccttcctgattt

SEQ ID NO: 50 - DNA, 23 bp, Homo sapiens
  source 1..23: mol_type = other DNA
ttctttagcactccttggcaaaa

SEQ ID NO: 51 - DNA, 20 bp, Homo sapiens
  source 1..20: mol_type = other DNA
ccaatggtgctcctggtatt

SEQ ID NO: 52 - DNA, 19 bp, Homo sapiens
  source 1..19: mol_type = other DNA
gttcaccgctgttaccctt

SEQ ID NO: 53 - DNA, 24 bp, Homo sapiens
  source 1..24: mol_type = other DNA
attattttggcacaacaggaagct

SEQ ID NO: 54 - DNA, 22 bp, Homo sapiens
  source 1..22: mol_type = other DNA
tccgcataggactgaccaagat

SEQ ID NO: 55 - DNA, 20 bp, Homo sapiens
  source 1..20: mol_type = other DNA
accgaaatcacagccagtag

SEQ ID NO: 56 - DNA, 22 bp, Homo sapiens
  source 1..22: mol_type = other DNA
cctcctcactcagctcatattc

SEQ ID NO: 57 - DNA, 20 bp, Homo sapiens
  source 1..20: mol_type = other DNA
gccctctgtgccacagatgt

SEQ ID NO: 58 - DNA, 15 bp, Homo sapiens
  source 1..15: mol_type = other DNA
ccccgctgccaccat

SEQ ID NO: 59 - DNA, 25 bp, Homo sapiens
  source 1..25: mol_type = other DNA
ggactcttcggagaactttcagatc

SEQ ID NO: 60 - DNA, 19 bp, Homo sapiens
  source 1..19: mol_type = other DNA
gggcacaggaaaacatcga

SEQ ID NO: 61 - DNA, 20 bp, Homo sapiens
  source 1..20: mol_type = other DNA
cgggctttggacaccaatta

SEQ ID NO: 62 - DNA, 18 bp, Homo sapiens
  source 1..18: mol_type = other DNA
gggcgcacacagcagttc

SEQ ID NO: 63 - DNA, 22 bp, Homo sapiens
  source 1..22: mol_type = other DNA
gaaccaacggagaaaaaaatgg

SEQ ID NO: 64 - DNA, 18 bp, Homo sapiens
  source 1..18: mol_type = other DNA
gggagggtgggtcttgga

SEQ ID NO: 65 - DNA, 19 bp, Homo sapiens
  source 1..19: mol_type = other DNA
ggttcggagacggcaaatt

SEQ ID NO: 66 - DNA, 19 bp, Homo sapiens
  source 1..19: mol_type = other DNA
ggaacgcactgcaaaacga

SEQ ID NO: 67 - DNA, 18 bp, Homo sapiens
  source 1..18: mol_type = other DNA
cctggctgctctcaacat

SEQ ID NO: 68 - DNA, 22 bp, Homo sapiens
  source 1..22: mol_type = other DNA
cggatctccaagtactcaaagt

SEQ ID NO: 69 - DNA, 24 bp, Homo sapiens
  source 1..24: mol_type = other DNA
ttcccctcatcaagagctatctgt

SEQ ID NO: 70 - DNA, 20 bp, Homo sapiens
  source 1..20: mol_type = other DNA
acccgatgagaatggcagaa

SEQ ID NO: 71 - DNA, 23 bp, Homo sapiens
  source 1..23: mol_type = other DNA
ggaggatcaccgagatgataaag

SEQ ID NO: 72 - DNA, 20 bp, Homo sapiens
  source 1..20: mol_type = other DNA
ttctgctgtgcccttatctg

SEQ ID NO: 73 - DNA, 20 bp, Homo sapiens
  source 1..20: mol_type = other DNA
tacatcgggccttgcaaata

SEQ ID NO: 74 - DNA, 21 bp, Homo sapiens
  source 1..21: mol_type = other DNA
tgtcctcatccctctcataca

SEQ ID NO: 75 - DNA, 24 bp, Homo sapiens
  source 1..24: mol_type = other DNA
cctcgtctctaaacagtggaataa

SEQ ID NO: 76 - DNA, 22 bp, Homo sapiens
  source 1..22: mol_type = other DNA
gcgtcctgaacagaatcatcta

(19) United States
(12) Patent Application Publication
(10) Pub. No.: US 2024/0415801 A1
(43) Pub. Date: Dec. 19, 2024

(54) Methods and Compositions for Treating Fibroids

(71) Applicant: Lundquist Institute for Biomedical Innovation at Harbor-UCLA Medical Center, Torrance, CA (US)

(72) Inventors: Omid Khorram, Rolling Hills, CA (US); Tsai-Der Chuang, Inglewood, CA (US)

(21) Appl. No.: 18/743,767

(22) Filed: Jun. 14, 2024

Related U.S. Application Data
(60) Provisional application No. 63/472,994, filed on Jun. 14, 2023.

(51) Int. Cl. A61K 31/277; A61P 15/00

(52) U.S. Cl. CPC A61K 31/277; A61P 15/00

(57) ABSTRACT

Methods of treating a subject for fibroid(s) are provided. Aspects of the methods include administering to the subject a nuclear factor-kappa B (NF-kB) inhibitor, e.g., Bay 11-7082, to treat the subject for fibroid(s). Also provided are compositions for use in practicing methods of the invention.

### CROSS REFERENCE TO RELATED APPLICATIONS

Pursuant to 35 U.S.C. § 119 (e), this application claims priority to the filing date of the U.S. Provisional Patent Application Ser. No. 63/472,994, filed Jun. 14, 2023; the disclosure of which application is herein incorporated by reference.

### ACKNOWLEDGEMENT OF GOVERNMENT RIGHTS

This invention was made with Government support under contract R01HD100529-01 awarded by the National Institutes of Health. The Government has certain rights in the invention.

### REFERENCE TO AN ELECTRONIC SEQUENCE LISTING

The contents of the electronic sequence listing (LABIO-007_SEQ_LIST.xml; Size: 67,572 bytes; and Date of Creation: Aug. 1, 2024) is herein incorporated by reference in its entirety.

### INTRODUCTION

Fibroid is a benign tumor of muscular and fibrous tissues, typically developing in the wall of the uterus. Uterine fibroids, also known as uterine leiomyomas, are benign smooth muscle tumors of the uterus. Most women have no symptoms while others may have painful or heavy periods. If large enough, they may push on the bladder causing a frequent need to urinate. They may also cause pain during sex or lower back pain. A woman can have one uterine fibroid or many. Occasionally, fibroids may make it difficult to become pregnant, although this is uncommon.

The exact cause of uterine fibroids is unclear. However, fibroids run in families and appear to be partly determined by hormone levels. Risk factors include obesity and eating red meat. Diagnosis can be performed by pelvic examination or medical imaging.

About 20% to 80% of women develop fibroids by the age of 50. In 2013, it was estimated that 171 million women were affected worldwide. They are typically found during the middle and later reproductive years. After menopause, they usually decrease in size. In the United States, uterine fibroids are a common reason for surgical removal of the uterus.

Treatments typically focus on symptoms. NSAIDs, such as ibuprofen, may help with pain and bleeding. Iron supplements may be needed in those with heavy periods. Medications of the gonadotropin-releasing hormone agonist class may decrease the size of the fibroids but are expensive and associated with side effects. If greater symptoms are present, surgery to remove the fibroid or uterus may help. Uterine artery embolization may also help. Cancerous versions of fibroids are rare and are known as leiomyosarcomas.

Other attempted therapies include those that try to reduce estrogen/progesterone. However, such therapies are problematic because of the associated side effects and can only be used for short period of time. There is therefore a need for safe drugs that do not affect estrogen/progesterone and can be used for long term therapy.

### SUMMARY

Methods of treating a subject for fibroid(s) are provided. Aspects of the methods include administering to the subject an inhibitor of nuclear factor-kappa B (NF-kB), e.g., Bay 11-7082, to treat the subject for fibroid(s). Also provided are compositions for use in practicing methods of the invention.

### BRIEF DESCRIPTION OF THE FIGURES

The invention may be best understood from the following detailed description when read in conjunction with the accompanying drawings. Included in the drawings are the following figures:

FIGS. 1A-1B. (A) Fresh fibroids explants were implanted subcutaneously in the ovariectomized CB-17 SCID/Beige mice and the vehicle or Bay 11-7082 (20 mg/kg/daily) were administered i.p daily for 8 weeks. The weights of the tumor explants were determined after 8 weeks of treatment (n=10). (B) Representative images of four xenografts at the end of treatment period (8 weeks). The results are presented as mean±SEM of independent experiments with P values indicated at the corresponding line. ***p<0.001.

FIGS. 2A-2E. (A) Hierarchical clustered heatmap analysis of the differentially expressed transcripts (fold change ≥1.5, p<0.05) in xenografts following 8 weeks of treatment of vehicle or Bay 11-7082 (n=4). Color gradient represents gene expression as z-scores. (B) Volcano plot showing up (n=374; red) and downregulated genes (n=629; blue) with a false discovery rate (FDR) p-value <0.05. (C) Heatmap of the 118 enriched hub genes identified by the CytoHubba plugin of the Cytoscape software platform. Color gradient represents gene expression levels as z-scores. (D) Gene ontology (GO) analysis of the 118 enriched hub genes. Color gradient represents levels of log 2 fold change presented as z-scores. (E) The Protein-Protein Interaction Networks of the 118 hub genes were constructed by the Search Tool for the Retrieval of Interacting Genes (STRING) database and Cytoscape software version 3.10.2. The color of nodes denotes interaction degree (from high to low degree: red, orange, and yellow).

FIG. 3. Relative expression of SPARC, TDO2, MYD88, TLR6, TLR3, IL6, IL8, TNFα, TNFRSF11A, IL1β, COL3A1, FN1, LOX, TGFB3, CCND1, E2F1, CKS2, PRL, ESR1, PGR, VEGFC, PDGFA and HTR1 B mRNA in xenografts implanted subcutaneously in the ovariectomized CB-17 SCID/Beige mice (n=10) following 8 weeks of treatment with vehicle or Bay 11-7082 (20 mg/kg/daily). The results are presented as mean±SEM with P values indicated by corresponding lines. *p<0.05; **p<0.01; ***p<0.001.

FIGS. 4A-4C. (A) Representative Western blot analysis of COL3A1, FN1, SPARC, TDO2 and Cleaved Caspase 3 with bar graphs (B) showing their relative band densities in the xenografts (n=10). (C) Total collagen levels as determined by enzyme-linked immunosorbent assay in 10 xenografts. The results are presented as mean±SEM of independent experiments with p values indicated at the corresponding line. *p<0.05; **p<0.01.

FIGS. 5A-5J. (A-H) Representative immunohistochemically stained images of fibroid xenografts treated with vehicle or Bay 11-7082 (magnification, ×20) for Ki67 (A-B), CCND1 (C-D), E2F1 (E-F) and cleaved caspase 3 (G-H). Quantification analysis by Halo software for the positive staining in nucleus (B, D, F and H; n=5 in each group). (I) Representative histopathological images determined by Masson's trichrome staining of fibroid xenografts from vehicle or Bay 11-7082-treated group (magnification, ×20). Blue color demonstrates collagen fibers and red color indicates smooth muscle cells. (J) Shows the quantification of staining intensity by Halo software (n=5 in each group). The results are presented as mean±SEM with P values indicated at the corresponding line. *p<0.05; **p<0.01.

FIG. 6. Relative expression of SPARC, TDO2, MYD88, TLR6, TLR3, IL6, IL8, TNFα, TNFRSF11A, IL1β, COL3A1, FN1, LOX, TGFB3, CCND1, E2F1, CKS2, PRL, ESR1, PGR, VEGFC, PDGFA and HTR1 B mRNA in fibroid explants (n=4) following 48 hours of treatment with vehicle or Bay 11-7082 (5 μM). The results are presented as mean±SEM with P values indicated by corresponding lines. *p<0.05; **p<0.01; ***p<0.001.

FIGS. 7A-7B. Relative expression of miR-29c and miR-200c in xenografts implanted subcutaneously in the ovariectomized CB-17 SCID/Beige mice (n=10) following 8 weeks of treatment with vehicle or Bay 11-7082 (20 mg/kg/daily) (A) or in fibroid explants (n=4) following 48 hours of treatment with vehicle or Bay 11-7082 (5 μM) (B). The results are presented as mean±SEM with P values indicated by corresponding lines. *p<0.05.

FIGS. 8A-8C. Correlation analysis between the fold change in tumor weight with levels of FN1 (A), MyD88 (B), and miR-29c (C) in the xenografts.

FIG. 9. Relative expression of IL8, TGFp3, Col3A1, CDK2, CCND1, DNMT1 and DNMT3A in 3D cultured leiomyoma smooth muscle cells (LSMC) treated with selective NF-kB inhibitor, Bay 11-7082 (5 μM) for 24 h. The results are presented as mean±SEM in three independent experiments using three different LSMC with control values independently set as 1 and analyzed using student t test with p values indicated by corresponding lines.

FIG. 10. Fresh fibroids explants were implanted subcutaneously in the ovariectomized CB-17 SCID/Beige mice and the vehicle or Bay 11-7082 (5 mg/kg/every other day) were administered i.p daily for 8 weeks. The weight of tumor explants were determined after 8 weeks of treatment (n=13). The results are presented as mean±SEM of independent experiments with P values indicated at the corresponding line. ***P<0.001.

FIG. 11. Relative expression of COL1A1, COL3A1, FN1, TGF-β3, CCND1, CDK2, E2F1, EZH2, MyD88 and SPARC mRNA in xenografts implanted subcutaneously in the ovariectomized CB-17 SCID/Beige mice (n=13) following 8 weeks of treatment with vehicle or Bay 11-7082 (5 mg/kg/every other day). The results are presented as mean±SEM with P values indicated by corresponding lines. *P<0.05; **P<0.01; ***P<0.001.

FIGS. 12A-12J. (A-B) Representative histopathological images determined by Masson's trichrome staining of fibroid xenografts from vehicle or Bay 11-7082-treated group (magnification, ×20). Blue color demonstrates collagen fibers and red color indicates smooth muscle cells. (B) Shows the quantification of staining intensity by Halo software (n=5 in each group). (C-J) Representative immunohistochemically stained images of fibroid xenografts treated with vehicle or Bay 11-7082 (magnification, ×20) for Ki67 (C-D), CCND1 (E-F), E2F1 (G-H) and cleaved caspase 3 (I-J). Quantification analysis by Halo software for the positive staining in nucleus (D, F, H) and cytosol (J) (n=5 in each group). The results are presented as mean±SEM with P values indicated at the corresponding line. *P<0.05; **P<0.01.

### DEFINITIONS

As used in the present specification, the following words, phrases and symbols are generally intended to have the meanings as set forth below, except to the extent that the context in which they are used indicates otherwise.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of fibroids. The therapeutically effective amount may vary depending on the subject, and disease or condition being treated, the weight and age of the subject, the severity of the disease or condition, and the manner of administering, which can readily be determined by one or ordinary skill in the art.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### DETAILED DESCRIPTION

Methods of treating a subject for fibroid(s) are provided. Aspects of the methods include administering to the subject a nuclear factor-kappa B (NF-kB) inhibitor, e.g., Bay 11-7082, to treat the subject for fibroid(s). Also provided are compositions for use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods of Treatment

As summarized above, methods of treating a subject for a fibroid(s) are provided. By treatment, is meant that at least an amelioration of the symptoms associated with the condition afflicting the host is achieved, where amelioration is used in a broad sense to refer to at least a reduction in the magnitude of a parameter, e.g., a symptom associated with the condition being treated or a side effect resulting from administration of a drug. As such, treatment also includes situations where the pathological condition, or at least symptoms associated therewith, are completely inhibited, e.g., prevented from happening, or stopped, e.g., terminated, such that the host no longer suffers from the condition, or at least the symptoms that characterize the condition. Treating also includes prophylactically treating the subject, such that the fibroid does not occur in the subject. As such, treating includes preventing the occurrence of the condition in the subject.

A variety of subjects are treatable according to the subject methods. Generally, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans.

In certain embodiments, the subjects will be subjects that have been diagnosed for and are, therefore, in need of administration of the active agent. In certain embodiments, the methods may include diagnosing the subject for the presence of the disease condition to be treated by administration of the active agent.

In accordance with one embodiment of the present disclosure, provided is a method of treating fibroid in a patient in need thereof, which entails administering to the patient an effective amount of a nuclear factor-kappa B (NF-kB) inhibitor, e.g., Bay 11-7082.

As provided, fibroid is a benign tumor of muscular and fibrous tissues, typically developing in the wall of the uterus. Uterine fibroids are benign smooth muscle tumors of the uterus. The type of fibroid developed depends on its location in or on the uterus.

Intramural fibroids are the most common type of fibroid. These types appear within the muscular wall of the uterus. Intramural fibroids may grow larger and can stretch a patient's uterus.

Subserosal fibroids form on the outside of the uterus, which is called the serosa. They may grow large enough to make a patient's uterus appear bigger on one side.

Fibroids can develop a stem, a slender base that supports the tumor. When this occurs, these fibroids are known as pedunculated fibroids. Pedunculated fibroids may grow on the outside of the uterus (e.g., subserosal) or on the inside of the uterus (e.g., submucosal).

Submucosal fibroids develop in the middle muscle layer, or myometrium, of the uterus. Submucosal fibroids are less common than other types of fibroids. However, submucosal fibroids distort the uterine cavity, and thus even small lesions in this location may lead to bleeding and infertility. A pedunculated lesion within the cavity is termed an intracavitary fibroid and can be passed through the cervix.

In one embodiment, the compositions and methods of the present disclosure are useful for treating intramural fibroids, subserosal fibroids, and submucosal fibroids. In one embodiment, the compositions and methods of the present disclosure are useful for treating one or more symptoms of fibroid. Examples of such symptoms include, without limitation, heavy bleeding between or during periods that includes blood clots, pain in the pelvis or lower back, increased menstrual cramping, increased urination, pain during intercourse, menstruation that lasts longer than usual, pressure or fullness in your lower abdomen, and swelling or enlargement of the abdomen.

Aspects of embodiments of the methods include administering to the subject a nuclear factor-kappa B (NF-kB) inhibitor, e.g., Bay 11-7082, to treat the subject for fibroids. Inhibitors of NF-kB employed in embodiments of the invention may thus include the following types (from downstream to upstream): DNA binding inhibitors including GYY 4137, p-XSC, CV 3988, and Prostaglandin E2 (PGE2) that inhibit the binding between NF-kB/Rel and its target DNA, thus inhibiting any gene expression activated by NF-kB; inhibitors of post-translational modifications on NF-kB/Rel, e.g. a p65 acetylation inhibitor, including Gallic acid and Anacardic acid that prevents NF-kB from activating its target genes; translocation inhibitors including JSH-23, and Rolipram that prevents NF-kB/Rel from translocating to the nucleus; IKB degradation inhibitors including BAY 11-7082, MG-115, MG-132, Lactacystin, Epoxomicin, Parthenolide, Carfilzomib, and MLN-4924 (Pevonedistat) that prevents ubiquitinated IKB from being degraded, thus maintaining IKB'S suppression of NF-kB/Rel functions; IKK inhibitors including TPCA 1, NF-KB Activation Inhibitor VI (BOT-64), BMS 345541, Amlexanox, SC-514 (GK 01140), IMD 0354, and IKK-16 that prevent the phosphorylation of IKB and thus preventing the ubiquitination and degradation of IKB. In some embodiments, NF-kB inhibitors are proteasome inhibitors including MG132, bortezomib, carfilzomib, and ixazomib. In some embodiments, NF-kB inhibitors inhibit nuclear translocation inhibitors including dehydroxymethylepoxyquinomicin (DHMEQ), small peptidomimetics, such as SN-50, which encompasses the NLS of p50. In some embodiments, NF-kB inhibitors inhibit NF-kB'S DNA binding, including sesquiterpene lactone (SL) compounds and decoy oligodeoxynucleotides.

Persons of skill in the art will readily recognize additional types of NF-kB inhibitors based on the mechanistic pathways involved, including, e.g., agents that can inhibit protein kinases, protein phosphatases, proteasomes, ubiquitination, acetylation, methylation, and DNA binding steps have been identified as NF-KB inhibitors. (Pires et al, Genes (Basel). 2018 Jan. 9; 9(1). pii: E24; Gupta, 2010 October-December; 1799(10-12):775-87).

The contents of Pires et al, Genes (Basel). 2018 Jan. 9; 9(1) and Gupta et al, Biochim Biophys Acta., 2010 October-December; I799(IO-I2) are hereby incorporated by reference. Table 1 of Gupta et al, Biochim Biophys Acta., 2010 October-December; 1799(10-12) lists some of the known NF-kB inhibitors. Persons of skill in the art will understand that the inhibitors may be small molecules, biologics, or other types of agents that block the function of NF-kB. In some embodiments, the NF-kB inhibitors are antibodies against targets affecting NF-kB functions. The antibodies may be blocking antibodies or agonistic antibodies depending on the involvement of the antibody's target in NF-kB functionality.

Non-limiting examples of NF-kB inhibitors also include I5d-PGJ(2), Calagualine, Conophylline, Evodiamine, Geldanamycin, Perrilyl alcohol, PSK, Rocaglamides, Adenovirus E1A, NS5A (Hep-C virus), Erbin overexpression, Golli BG21, KSR, MAST205, PEDF, Rituximab, TNAP, Betaine, Desloratadine, LY29 and LY30, MOL 294, Pefabloc, Rhein, SMI and FP, [6]-gingerol, I′-Acetoxychavicol acetate, 20(S)-Protopanaxatriol, 4-Hydroxynonenal, Acetyl-boswellic acids, Anandamide, Anethole, Apigenin, Artemisia vestital, Baoganning, Betulinic acid, Buddlejasaponin IV, Cacospongionolide B, Calagualine, Cardamomin, Casparol, Cobrotoxin, Cycloepoxydon, Decursin, Dehydroascorbic acid, Dexanabinol, Digitoxin, Diosgenin, Diterpenes, Docosahexaenoic acid, Falcarindol, Flavopiridol, Furonaphthoquinone, Garcinone B, Glycine chloramine, Guggulsterone, Herbimycin A, Honokiol, Hypoestoxide, Indirubin-3′-oxime, Isorhapontigenin, Clarithromycin, Cloricromene, C-K and Rh(2), Cryptotanshinone, Cytochalasin D, Danshenshu, Diterpenoids, Ent-kaurane diterpenoids, Epinastine hydrochloride, Epoxyquinol A, Erythromycin, Evodiamine, Fucoidan, Gallic acid, Ganoderma lucidum, Garcinol, Geranylgeraniol, Ginkgolide B, Glycyrrhizin, Halofuginone, Hematein, Herbal compound 861, Hydroxy ethyl starch, Hydroxy ethylpuerarin, Hypericin, Kamebakaurin, Linoleic acid, Lithospermi radix, Macrolide antibiotics, 2-methoxyestradiol, 6-MITC, Oridonin, Plant compound A, Polyozellin, Prenylbisabolane 3, Prostaglandin E2, PSK, Quinic acid, Sanggenon C, Sesamin, Shen-Fu, Sibbinin, Sinomenine, Tansinones, Taurine+niacine, TZD MCC-555, Trichostatin A, Triptobde, Tyrphostin AG-126, Ursolic acid, Withaferin A, Xanthohumol, Xylitol, Yan-gan-wan, Yin-Chen-Hao, Ghrelin, Peptide YY, Rapamycin, Adiponectin, Kahweol, Manumycin A, Monochloramine, N-acetylcysteine, Nitric oxide, Nitrosylcobalamin, Oleandrin, Omega 3 fatty acids, ox-LDL, Panduratin A, PEITC, Petrosaspongiolide M, Phytic acid, Piceatannol, Pinosylvin, Plumbagin, Prostaglandin Al, Quercetin, Rengyolone, Rosmarinic acid, Rottlerin, Saikosaponin-d, Sanguinarine, Staurosporine, Sesquiterpene lactones, Scoparone, Silibinin, Silymarin, Sulforaphane, Sulindac, Tetrandine, Theaflavin, Thienopyridine, Tibanin, Ursolic acid, Vesnarinone, Wedelolactone, Withanobdes, Xanthoangelol D, Zerumbone, b-carboline, g-mangostin, g-Tocotrienol, IKKb peptide, NEMO CC2-LZ peptide, Anti-thrombin III, Chorionic gonadotropin, FHIT, HB-EGF, Hepatocyte growth factor, Interferon-a, Interleukin-10, PAN1, PTEN, SOCS1, Adenovirus, MC159, MC160, Angiopoietin-1, Antithrombin, b-catenin, Bromelain, CaMKK, CD43 overexpression, FLN29 overexpression, FLIP, G-120, Interleukin 4, Transdominant p50, VEGF, ADP ribosylation inhibitor, 7-amino-4-methylcoumarin, Amrinone, Atrovastat, Benfotiamine, Benzamide, Bisphenol A, Caprofen, Carbocisteine, Celecoxib, Germcitabine, Cinnamaldehyde, 2-methoxy CNA, 2-hydroxy CNA, CDS, CP Compound, Cyanoguanidine, HMP, a-difluoromethylomithine, DTD, Evans Blue, Evodiamine, Fenoldopam, FEX, Fibrates, FK778, Flunixin meglumine, Flurbiprofen, Hydroquinone, IMD-0354, JSH-21, KT-90, Lovastatin, Mercaptopyrazine, Mevinobn, Monoethylfumarate, Moxifloxacin, Nicorandil, Nilvadipine, NO-ASA, Panepoxy done, Peptide nucleic acids, Perindopril, PAD, a-PBN, Pioglitazone, Pirfenidone, PNO derivatives, Quinadril, AIDCA derivative, TDZD, TPCA-1, Pyridine derivatives, ACHP, Acrolein, AGRO100, Amino-pyrimidine, AS602868, Aspirin, Azidothymidine, BAY-I 1-7082, BAY-I 1-7083, Benzoimidazole derivative, Benzyl isothiocyanate, BMS-345541, Carboplatin, CDDO-Me, CHS 828, Compound 5, Compound A, Cyclopentenones, CYL-19s, CYL-26z, Diaylpyridine derivative, DPE, Epoxyquinone, Gabexate mesilate, Gleevec, Hydroquinone, Ibuprofen, IQCAD, Indolecarboxamide, Isobutyl nitrite, Jesterone dimer, 15-deoxyspergualine analog, Methotrexate, MLB 120, Monochloramine, MX781 (Retinoid antagonist), 4-HPR, Nafamostat mesilate, NSAIDs, PS-1145 (MLN1145), PQD, Pyridooxazinone derivative, SC-514, Scytonemin, Sodium salicylate, Statins (several), Sulfasalazine, Sulfasalazine analogs, Survanta, Thalidomide, THI 52, YC-I, Lead, Mild hypothermia, Saline (low Na+), 5′-methylthioadenosine, Alachlor, Amentoflavone, Antrodia camphorata, Aucubin, Baicalein, Raxofelast, Ribavirin, Rifamides, Ritonavir, Rosiglitazone, Roxithromycin, DAAS, Serotonin derivative, Simvastatin, SM-7368, T-614, Sulfasalazine, SUN C8079, Triclosan plus CPC, Tobacoo smoke, Verapamil, Hypercapnic acidosis, Hyperosmolarity, Hypothermia, Alcohol, 4′-DM-6-Mptox, 4-phenylcoumarins, AHUP, Luteolin, Mesuol, Nobiletin, Phomol, Psychosine, Qingkailing, Saucemeol D & E, Shuanghuanglian, Trilinolein, Wortmannin, a-zearalenol, NF-kappaB-repression factor, PIAS3, PTX-B, 17-AAG, TMFC, AQC derivatives, 9-aminoacridine derivatives, Chromene derivatives, D609, Dimethylfumarate, EMDPC, Histidine, Mesalamine, PEITC, Pranlukast, R031-8220 (PKC, inhibitor), SB203580 (MAPK inhibitor), Tetrathiomolybdate, Tranilast, Troglitazone, Catalposide, Cyclolinteinone, Dihydroarteanniun, Docosahexaenoic acid, Emodin, Ephedrae herba (Mao) extract, Equol, Erbstatin, Ethacrynic acid, Fosfomycin, Genipin, Genistein, Glabridin, Glucosamine sulfate, Isomallotochromanol, Isomallotochromene, Melatonin, Midazolam, Momordin I, Polymyxin B, Prostaglandin, Resiniferatoxin, Thiopental, Tipifamib, TNP-470, Ursodeoxycholic acid, b-PEITC, 8-MSO, b-lapachone, Penetratin, VIP, Activated protein C, HSP-70, Interleukin-13, Intravenous Ig, Murrl gene product, Neurofibromatosis-2 protein, PACAP, SAIF, a-MSH, g-glutamylcysteine synthetase, I-Bromopropane, Acetaminophen, Diamide, Dobutamine, Cyclosporin A, Lactacystine, b-lactone, APNE, Boronic acid peptide, BTEE, 3,4-dichloroisocoumarin, Deoxyspergualin, DFP, Disulfiram, FK506 (Tacrolimus), Bortezomib, Salinosporamide A, 23-hydroxyursolic acid, Anetholdithiolthione, Apocynin, Arctigenin, Aretemisa p7F, Astaxanthin, Benidipine, bis-eugenol, BG compounds, BHA, CAPE, Camosol, Carvedilol, Catechol derivatives, Celasterol, Cepharanthine, Chlorogenic acid, Chlorophyllin, Curcumin, DHEA, DHEA sulfate, Dehydroevodiamine, Demethyltraxillagenin, Diethyldithiocarbamate, Diferoxamine, Dihydroisoeugenol, Dihydrolipoic acid, Dilazep, Fenofibric acid, DMDTC, Dimethylsulfoxide, Disulfiram, Ebselen, Edaravone, EGTA, EPC-K1, Epigallocatechin-3-gallate, Ergothioneine, Ethyl pyruvate, Garcinol, g-glutamylcysteine synthetase, Glutathione, Hematein, Hydroquinone, Hydroquinone, IRFI 042, Iron tetrakis, Isovitexin, Kangen-karyu extract, Ketamine, Lacidipine, Lazaroids, L-cysteine, Lupeol, Magnolol, Maltol, E-73, Ecabet sodium, Gabexate mesilate, Glimepiride, Hypochlorite, Losartin, LY294002, Pervanadate, Phenylarsine oxide, Phenytoin, Rol06-9920, Sabaeksan, U0126 (MEK inhibitor), 15-deoxyspergualin, 2′,8″-biapigenin, 5F (from Pteri syeminpinnata), Alginic acid, Apigenin, Astragaloside IV, AT514 (serratamolide), Atorvastatin, Cantharidin, Chiisanoside, Clarithromycin, Eriocalyxin B, Hirsutenone, JM34, KIOM-79, Leptomycin B, Neomycin, Nucling, Oregonin, OXPAPC, Paeoniflorin, Phallacidin, Piperine, Pitavastatin, Rapamycin, Selenomethionine, Shenfu, Sopoongsan, Sphondin, T. poly glycosides, Younggaechulgam-tang, a-pinene, NCPP, PN50, Mangiferin, Melatonin, Mn-SOD, Myricetin, N-acetyl-L-cysteine, Nacyselyn, Naringin, N-ethyl-maleimide, Nitrosoglutathione, NDGA, Ochnaflavone, Orthophenanthroline, Phenylarsine oxide, Pyrithione, Pyrrolinedithiocarbamate, Quercetin, Quinozolines, Rebamipide, Redox factor 1, Resveratrol, Rotenone, Roxithromycin, S-allyl-cysteine, Sauchinone, Sodium 4-Aminosalicylate, Spironolactone, Taxifolin, Tempol, Tepoxaline, tert-butyl hydroquinone, Tetracylic A, Wogonin, xanthohumol, Yakuchinone A, B, a-lipoic acid, a-tocopherol, a-torphryl acetate, a-torphryl succinate, b-Carotene, Diltiazem, Dioxin, Dipyridamole, Disulfiram, Enalapril, Fluvastatin, Indole-3-carbinol, JSH-23, KL-1156, Leflunomide, Levamisole, Moxifloxacin, Omapatrilat, R-etodolac, Rolipram, SC236 (COX-2 inhibitor), Triflusal, Actinodaphine, Artemisinin, Baicalein, b-lapachone, Calcitriol, Campthothecin, Capsiate, and Catalposide. See, e.g., Gupta et al, Biochim Biophys Acta., 2010 October-December; I799(IO-I2). In some embodiments, the NF-kB inhibitor is Andrographolide; Bay 11-7082; Bithionol; Bortezomib; CBL0137 (CBL-0137); Cantharidin; Chromomycin A3; Daunorubicinum; Diethylmaleate; Digitoxin; Ectinascidin 743; Emetine; Evodiamine (Isoevodiamine); Fluorosalan; GSK2982772; GSK583; Indole-3-carbinol; JSH-23; Magnolol; Manidipine hydrochloride; Narasin; Lestaurtinib; Omaveloxolone (RTA-408); Ouabain; QNZ (EVP4593); (−)-Parthenolide; Pyrrolidinedithiocarbamate ammonium; Rapamycin; SC75741; Sorafenib tosylate; Sunitinib malate; Tioconazole; Tribromsalan; Triclabendazolum; Triptolide (PG490); or Zafirlukast. In some embodiments, the inhibitor is emetine, fluorosalan, sunitinib malate, bithionol, narasin, tribromsalan, lestaurtinib, ectinascidin 743, chromomycin A3, or bortezomib. See, e.g., Miller et al, Biochem Pharmacol. 2010 May 1; 79(9): 1272-1280. In some embodiments, the NF-kB inhibitor is not sodium salicylate.

In some embodiments, the NF-kB inhibitor is an inhibitor of IKK. IKK comprises two subunits IKKa and IKKb. Each subunit is important for the phosphorylation of IKB. (Mazhar Adli, IKKa and IKKb Each Function to Regulate NF-kB Activation in the TNF-Induced/Canonical Pathway, PLoS One. 2010; 5(2): e9428). In some embodiments, an additional component of IKK is IKKg/NEMO. In some embodiments, the NF-kB inhibitor inhibits the function of IKKa; in some embodiments, the NF-kB inhibitor inhibits the function of IKKb; and in some embodiments, the NF-kB inhibitor inhibits both the function of IKKa and the function of IKKb. In some embodiments, the NF-kB inhibitor inhibits IKKg/NEMO. IKK inhibitors can include ATP analogs, allosteric modulators, and agents interfering with the kinase activation loops. (Begalli et. al, Unlocking the NF-kB Conundrum: Embracing Complexity to Achieve Specificity, Biomedicines. 2017 Aug. 22; 5(3). pii: E50). Examples of ATP analogues include b-carboline, SPC-839, BMS-345541, and SAR-I 13945. In some embodiments, the NF-kB inhibitor is Bay 11-7082. Bay 11-7082 inhibits both IKKa and the function of IKKb. (Rauert-Wunderlich, The IKK inhibitor Bay 11-7082 induces cell death independent from inhibition of activation of NF-kB transcription factors, PLoS One. 2013; 8(3):e59292). Other known IKK inhibitors include IMD-0354 (N-(3,5-Bis-trifluoromethylphenyl)-5-chloro-2-hydroxybenzamide), TPCA 1, NF-KB Activation Inhibitor VI (BOT-64), BMS 345541, Amlexanox, SC-514 (GK 01140), IMD 0354, and IKK-16. The contents of Begalli F et. al, Unlocking the NF-kB Conundrum: Embracing Complexity to Achieve Specificity, Biomedicines. 2017 Aug. 22; 5(3). pii: E50, and Rauert-Wunderlich et. al, The IKK inhibitor Bay 11-7082 induces cell death independent from inhibition of activation of NF-kB transcription factors, PLoS One. 20I3; 8(3):e59292 are hereby incorporated by reference.

In some embodiments, the NF-kB inhibitor is an NIK inhibitor. NIK inhibitors include, but are not limited to, alkynyl alcohols (as disclosed in WO2009158011); 6-membered heteroaromatic substituted cyanoindoline derivatives (as disclosed in WO2017125534); pyrazoloisoquinoline derivatives (as disclosed in JP2017031146); the compounds disclosed in FIG. 14 of WO2013014244; 6-azaindole aminopyrimidine derivatives (as disclosed in US20110183975); a polypeptide that blocks NIK-HC8 binding (as disclosed in U.S. Pat. No. 8,338,567); pyrazoloisoquinoline derivatives (as disclosed in U.S. Pat. No. 6,841,556); candidate inhibitors listed in Table 1 of Wang et al., including sulfapyridine and propranolol (as disclosed in Wang et al. (2018). Sci Report, 8: 1657); tricyclic NF-KB inducing kinase inhibitors (as disclosed in Castanedo et el. (2017). J Med Chem, 60 (3): 627-640, e.g., compound 10 (9-fluoro-10-[(3R)-3-hydroxy-3-(5-methylisoxazol-3-yl)but-1-ynyl]-5,6-dihydroimidazo[1,2-d][1,4]benzoxazepine-2-carboxamide), 32 (10-fluoro-9-[(3R)-3-hydroxy-3-(5-methyl-1,2-oxazol-3-yl)but-1-yn-1-yl]-2,5-diazatetracyclo[11.1.1.02,6.07,12]pentadeca-3,5,7(12),8,10-pentaene-4-carboxamide), or 33 (10-[(3R)-3-hydroxy-3-(5-methylisoxazol-3-yl)but-1-ynyl]-N3-methyl-5,6-dihydroimidazo[1,2-d][1,4]benzoxazepine-2,3-dicarboxamide); 4H-isoquinoline-1,3-dione and 2,7-naphthydrine-1,3,6,8-tetrone (as disclosed in Mortier et al. (Mortier et al. (2010). Bioorganic & Medicinal Chemistry Letters, 20 (15): 4515-4520)); and/or N-Acetyl-3-aminopyrazoles (as disclosed in Pippione et al. (2018). Medchemcomm. 9(6): 963-968). NIK inhibitors available from commercial suppliers, include, but are not limited to NIK-SMI1 ((R)-6-(3-((3-hydroxy-1-methyl-2-oxopyrrolidin-3-yl)ethynyl)phenyl)-4-methoxypicolinamide, Cat. No. PC-62514, ProbeChem), AM-0216 ((R)-4-(1-(2-aminopyrimidin-4-yl)indolin-6-yl)-2-(thiazol-2-yl)but-3-yn-2-ol, Cat. No. PC-35550. ProbeChem), AM-0561 ((R)-4-(3-(2-amino-5-chloropyrimidin-4-yl)imidazo[1,2-a]pyridin-6-yl)-2-(thiazol-2-yl)but-3-yn-2-ol, Cat. No. PC-35549, ProbeChem), or Amgen16 (1-((1-(2-amino-5-chloropyrimidin-4-yl)indolin-6-yl)ethynyl)cyclopentan-1-ol, Cat. No. PC-35548, ProbeChem).

Methods of identifying NF-kB inhibitors are known in the art (see e.g., Miller, et al. Identification of known drugs that act as inhibitors of NF-kappa B signaling and their mechanism of action. Biochem Pharmacol. 2010 May 1; 79(9):1272-80).Various NF-kB inhibitors are readily available through public sources. For example, Santa Cruz Biotechnology provides NF-kB inhibitors for purchases, including BAY 11-7085, Helenalin, NFkappaB Activation Inhibitor II, JSH-23, QNZ (EVP4593), Andrographolide, etc. (Santa Cruz Biotechnology). Various anti-NF-kB antibodies, for example, are available for purchase at Sigma-Aldrich, as well as antibodies against other proteins involved in NF-kB functionality, e.g., anti-IKK antibodies available at Sigma-Aldrich.

Aspects of the embodiments include methods of diagnosing fibroids in a patient. In some embodiments, the method is a method of treating fibroid in a patient who has been diagnosed with fibroid, comprising administering to the patient an effective amount of a nuclear factor-kappa B (NF-kB) inhibitor. Fibroids may be diagnosed via pelvic exam, ultrasound (e.g., transvaginal ultrasound or abdominal ultrasound), magnetic resonance imaging (MRI), computed tomography, laparoscopy, hysterosonography, hysterosalpingography, hysteroscopy, or other methods.

Fibroids and symptoms of fibroids may be measured using any of the following: fibroid size (e.g., volume, weight, dimensions), amenorrhea (PBAC score), myoma and uterine volumes, hemoglobin (Hb), Numerical Rating Scale (NRS) score, Uterine Fibroid Symptom and Quality of Life (UFS-QOL) scores, luteinizing hormone (LH), follicle-stimulating hormone (FSH), estradiol (E2) and progesterone (P), and combinations thereof.

The majority of the volume of fibroids usually consists of fibronectin, collagen and/or proteoglycan. Therefore, the control, treatment and/or prevention of fibroids can be measured by the release of fibronectin, collagen and/or proteoglycan. In particular, keeping approximately constant, decreasing, inhibiting, and/or preventing the release of fibronectin, collagen and/or proteoglycan are indicative of fibroid control, treatment and/or prevention.

Aspects of the embodiments include methods of treating fibroids with minimal to no adverse effects (e.g., minimal to no drug toxicity). In other words, the method of treatment is well tolerated. In some embodiments, the method is a method of treating fibroid in a patient, comprising administering to the patient an effective amount of nuclear factor-kappa B (NF-kB) inhibitor with minimal to no adverse effects. In other words, the treatment is safe. Treatment safety (e.g., drug safety) may be measured by comparing blood chemistry markers before and after treatment. Blood chemistry markers include, without limitation, general metabolism markers (e.g., glucose levels), kidney function markers (e.g., BUN levels, creatinine levels), electrolyte markers (e.g., sodium levels, phosphorous levels), liver function markers (e.g., alkaline phosphatase levels, albumin levels, SGPT levels, total protein levels, globulin levels, total bilirubin levels) and pancreas function markers (e.g., amylase levels). Minimal to no adverse effects may be defined by any of the above blood chemistry markers staying within the normal range for the subject after treatment. Minimal to no adverse effects may be quantified by a change in any of the above blood chemistry markers that is 90% or less after treatment compared to before treatment (including a change that is 80% or less, 70% or less, 60% or less, 50% or less, 40% or less, 30% or less, 20% or less, 15% or less, 10% or less, 5% or less, 2% or less, or 1% or less after treatment compared to before treatment).

“In combination with”, “combination therapy” and “combination products” refer, in certain embodiments, to the concurrent administration to a patient of the engineered proteins and cells described herein in combination with additional therapies, e.g., surgery, radiation, administration of another drug, and the like. When administered in combination, each component can be administered at the same time or sequentially in any order at different points in time. Thus, each component can be administered separately but sufficiently closely in time so as to provide the desired therapeutic effect.

In some embodiments, methods of treating fibroid in a patient may comprise administering to the patient an effective amount of a nuclear factor-kappa B (NF-kB) inhibitor in combination with one or more additional therapies (e.g., one additional therapy, two additional therapies, three additional therapies, four additional therapies, or five additional therapies). Additional therapies include, without limitation, gonadotropin-releasing hormone agonists, levonorgestrel-releasing intrauterine system, nonsteroidal anti-inflammatory drugs, oral contraceptives, progesterone receptor modulators, tranexamic acid, myolysis, myomectomy and uterine artery embolization. Gonadotropin-releasing hormone agonists (GnRH agonists) include, without limitation, abarelix, cetrorelix, degarelix, elagolix, ganirelix, linzagolix and relugolix. Levonorgestrel-releasing intrauterine systems (i.e., hormonal intrauterine devices (IUDs)) include, without limitation, Mirena, Skyla, Kyleena and Liletta. Nonsteroidal anti-inflammatory drugs (NSAIDs) include, without limitation, Aspirin, Diclofenac, Diflunisal, Etodolac, Fenoprofen, Flurbiprofen, Ibuprofen, Indomethacin, Ketoprofen, Ketorolac, Mefenamic acid, Meloxicam, Nabumetone, Naproxen, Oxaprozin, Piroxicam, Sulindac and Tolmetin. Oral contraceptives include, without limitation, estrogen-progesterone pills and progesterone-only pills. Estrogen-progesterone pills may comprise any combination of ethinylestradiol, norethindrone, norgestimate, drospirenone, levomefolate, norgestrel, desogestrel, levonorgestrel and estetrol. Progesterone-only pills may comprise norethindrone, drospirenone or norgestrel. Progesterone receptor modulators include, without limitation, ulipristal acetate, asoprisnil and telapristone.

### Compositions

Also provided are compositions for practicing the methods are described in the present disclosure. In general, subject compositions may have an NF-kB inhibitor, e.g., Bay 11-7082, in addition to a pharmaceutically acceptable excipient. In some embodiments, the subject compositions contain a secondary agent for treating any of the diseases or conditions described above.

Compositions of the present disclosure can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intra-epidural, or others as well as oral, nasal, ophthalmic, rectal, or topical. Sustained release administration is also specifically included in the disclosure, by such means as depot injections or erodible implants.

As noted above, active agents can be formulated with a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

The composition may be administered in a unit dosage form and may be prepared by any methods well known in the art. Such methods include combining agent with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Depending on the individual and condition being treated and on the administration route, the active agent may be administered in dosages of 0.01 mg to 500 mg/kg body weight per day, e.g., about 20 mg/day for an average person. Dosages will be appropriately adjusted for pediatric formulation.

In some embodiments, the composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the composition may further include a preservative. Suitable preservatives include, but are not limited to, a benzyl alcohol, phenol, chlorobutanol, benzalkonium chloride, and the like. In many cases, the composition is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

As described above, the composition may also contain a secondary agent for treatment of any of the diseases or conditions described above.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

The following is offered by way of illustration and not by way of limitation.

### EXAMPLES

### Example 1

### I. Introduction

Fibroids are benign tumors affecting a significant number of women causing pain, abnormal uterine bleeding, and infertility [1]. The pathogenesis of these tumors whose growth is regulated by ovarian steroids has been under intense investigation with inflammation playing a pivotal role in this process [2].

NF-Kb is a master regulator of inflammatory responses in fibroid pathogenesis. Aberrant NF-kB signaling has been implicated in a multitude of disorders including inflammatory, and autoimmune disorders and cancer. The NF-kB complex is composed of a family of inducible transcription factors including NF-kB1 (p50), NF-kB2 (p52), ReIA (p65), relB, and c-Rel. These transcription factors bind to a specific DNA site called kB enhancer as homo or hetero dimers inducing transcription of pro-inflammatory genes and are normally sequestered in the cytoplasm by a family of inhibitory IKB proteins [8]. The activation of NF-kB involves both the canonical and non-canonical pathways. The canonical pathway activation results in quick and transient transcription activity whereas the activation of the non-canonical pathway is slower and occurs through several TNF receptor superfamily members [8, 9]. Activation of NF-kB in fibroids may be evidenced by elevation of phosphorylated p65 and nuclear translocation of Rela:p65; this activation of NF-kB may lead to aberrant expression of miR-29c which regulates a wide range of ECM genes which are highly overexpressed in fibroids. Increased phosphorylation of IKBKB in fibroids induces phosphorylation of IkBa and its proteasome degradation, thereby allowing NF-kB dissociation and its nuclear translocation, showing that miR-200c which is also aberrantly expressed in fibroids regulated IL8 by targeting IKBKB. Here, Bay 11-7082 (Bay), an inhibitor of NF-kB is investigated for use in treating fibroids by inhibiting the expression of pro-inflammatory genes and inhibiting their growth. Bay was administered over a two-month course in a human fibroid xenograft mouse model and its effects on tumor weight and gene expression were examined. Bay is a phenyl vinyl sulfone that inhibits the NF-kB pathway by blocking the kinase activity of IKKB and inhibiting the NLRP3 inflammasome [13, 14]. It is also an inhibitor of protein tyrosine phosphatases and an inhibitor of p65 phosphorylation [14]. Bay has broad anti-inflammatory effects in various disease models [14] and induces apoptosis in various cell types including cancer cells [15, 16].

### II. Materials and Methods

### Fibroid Specimens Collection.

Portions of intramural uterine fibroids (2-5 cm diameter) were obtained from hysterectomies performed at Harbor-UCLA Medical Center for symptomatic fibroids (n=10). Prior approval was secured from the Institutional Review Board (18CR-31752-01 R) at the Lundquist Institute. Tissues were exclusively sourced from premenopausal patients who had not been treated with hormonal medications for at least 3 months before surgery. The fibroids used in this study were sourced from African American (n=2) and Hispanic (n=8) women aged between 35-50 years (mean 43±4.8 years). The MED12 mutation status of all fibroids was determined by PCR amplification and Sanger sequencing (Laragen Inc., Culver City, CA, USA). Among the tumors examined, five were found to harbor missense mutations in MED12 exon 2, specifically: c.130G>A (p.Gly44Ser) (n=1), c.131G>A (p.Gly44Asp) (n=2), c.130G>T (p.Gly44Cys) (n=1), and c.131G>T (p.Gly44Val) (n=1). Informed consent was obtained from all participants. A segment of each tumor was allocated for in vivo studies, and the remaining tissues were promptly snap-frozen and stored in liquid nitrogen for further analysis as previously described. This selection process for tumors was aimed at minimizing variance among fibroids [17-19].

### Fibroid Animal Model.

The protocol (31162-02) received approval from the IACUC at the Lundquist Institute, Harbor-UCLA Medical Center. Female ovariectomized SCID/Beige mice (Charles River Laboratories, Hollister, CA, USA), aged 9-12 weeks, were implanted with pellets (Innovative Research of America, Sarasota, FL, USA) containing estradiol (0.075 mg/60-day release) and progesterone (75 mg/60-day release) as previously described [20]. A portion of fresh fibroid weighing 0.5 g was sectioned aseptically into 5-10 pieces using a razor blade. Equal-weighted explants from the same patient were implanted in the flank of mice, which were then treated with Bay or vehicle, allowing for comparison of each treated tumor to its own control. Following 3 days of recovery, mice were injected daily with either vehicle (1% DMSO) or Bay (20 mg/kg) intraperitoneally. The dosage of Bay was determined based on prior studies of its efficacy in a mouse model of adult T-cell leukemia (ATL)/lymphoma [21]. After two months all mice were anesthetized with Ketamine (100 mg/kg) plus Xylazine (10 mg/kg) via intraperitoneal injection, followed by cardiac puncture for blood collection and cervical dislocation for euthanasia. Xenografts were dissected free of surrounding tissue, weighed, and frozen. Animals were weighed before and after treatment with Bay or vehicle. Surgical procedures and experiments were conducted in the C.W. Steers Biological Resources Center (BRC) animal care facility at the Lundquist Institute.

### Blood Chemistry Panel.

After 8 weeks of treatment with either the vehicle or Bay, plasma chemistry was analyzed. Specifically, 100 μL of plasma was transferred into the Comprehensive Diagnostic Profile Rotor (#500-1038, Abaxis, Union City, CA, USA). Subsequently, the levels of glucose, creatinine, BUN, phosphorus, sodium, albumin, alkaline phosphatase, serum glutamic pyruvic transaminase (SGPT; ALT), total protein, globulin, total bilirubin, and amylase were assessed using the VetScan VS2 chemistry analyzer (Abaxis, Union City, CA, USA) as previously described [20, 22].

### RNA Sequencing and Bioinformatic Analysis.

Total RNA was extracted from leiomyoma and matched myometrium using TRIzol (Thermo Fisher Scientific Inc., Waltham, MA). RNA concentration and integrity were assessed using a Nanodrop 2000c spectrophotometer (Thermo Scientific, Wilmington, DE) and Agilent 2100 Bioanalyzer (Agilent Technologies, Santa Clara, CA) as previously described. Samples with RNA integrity numbers (RIN) equal to or greater than 9 were selected for library preparation. One microgram of total RNA from each tissue was utilized to generate strand-specific cDNA libraries following the TruSeq protocol (Illumina, San Diego, CA). The RNA sequencing and analysis were carried out at UCLA Technology Center for Genomics & Bioinformatics. Flaski facilitated the visualization of differential gene expression through Hierarchical clustering, TreeView graphs, Volcano plots, and Pathway Enrichment Analysis [23]. Protein-Protein Interaction Networks were constructed using the Search Tool for the Retrieval of Interacting Genes (STRING) database [24]. Overall, the obtained RNA sequencing data met the criteria for subsequent statistical analysis. The RNA sequencing data has been deposited in the Gene Expression Omnibus (GEO) database under the accession number.

### Quantitative RT-PCR.

Quantitative RT-PCR was performed as previously described [19]. Selected gene expression levels were assessed utilizing the Invitrogen StepOne System, with normalization against 18S. Triplicate reactions were conducted, and relative mRNA expression was calculated using the comparative cycle threshold method (2−ΔΔCt), with results presented as fold change relative to the control group. The primer sequences used are shown in the following Sequence Table:

| | | SEQ ID |
|---|---|---|
| Primers | Sequences | NO |
| 18S (Forward) | 5′-CGAGCCGCCTGGATACC-3′ | 1 |
| 18S (Reverse) | 5′-CAGTTCCGAAAACCAACAAAATAGA-3′ | 2 |
| COL3A1 (Forward) | 5′-ATTATTTTGGCACAACAGGAAGCT-3′ | 3 |
| COL3A1 (Reverse) | 5′-TCCGCATAGGACTGACCAAGAT-3′ | 4 |
| FN1 (Forward) | 5′-ACCGAAATCACAGCCAGTAG-3′ | 5 |
| FN1 (Reverse) | 5′-CCTCCTCACTCAGCTCATATTC-3′ | 6 |
| LOX (Forward) | 5′-GATTGAGTCCTGGCTGTTATGA-3′ | 7 |
| LOX (Reverse) | 5′-GGGTTTACACTGACCTTTAGGAT-3′ | 8 |
| TGF-β3 (Forward) | 5′-CGGGCTTTGGACACCAATTA-3′ | 9 |
| TGF-B3 (Reverse) | 5′-GGGCGCACACAGCAGTTC-3′ | 10 |
| CCND1 (Forward) | 5′-GCCCTCTGTGCCACAGATGT-3′ | 11 |
| CCND1 (Reverse) | 5′-CCCCGCTGCCACCAT-3′ | 12 |
| E2F1 (Forward) | 5′-GGACTCTTCGGAGAACTTTCAGATC-3′ | 13 |
| E2F1(Reverse) | 5′-GGGCACAGGAAAACATCGA-3′ | 14 |
| CKS2 (Forward) | 5′-CATCTGATGTCTGAAGAGGAGTG-3′ | 15 |
| CKS2 (Reverse) | 5′-TATGTGGTTCTGGCTCATGAAT-3′ | 16 |
| SPARC (Forward) | 5′-TACATCGGGCCTTGCAAATA-3′ | 17 |
| SPARC (Reverse) | 5′-TGTCCTCATCCCTCTCATACA-3′ | 18 |
| TDO2 (Forward) | 5′-GCGATCAACTGTGAGTGATAGG-3′ | 19 |
| TDO2 (Reverse) | 5′-GGTTGGGTTCATCTTCGGTATC-3′ | 20 |
| MyD88 (Forward) | 5′-CCTGGCTGCTCTCAACAT-3′ | 21 |
| MyD88 (Reverse) | 5′-CGGATCTCCAAGTACTCAAAGT-3′ | 22 |
| TLR6 (Forward) | 5′-CCTGCCATCCTATTGTGAGTT-3′ | 23 |
| TLR6 (Reverse) | 5′-AGCTTCATAGCACTCAATCCC-3′ | 24 |
| TLR3 (Forward) | 5′-GACTCAGGTACCCGATGATCTA-3′ | 25 |
| TLR3 (Reverse) | 5′-GCTGGCTATACCTTGTGAAGT-3′ | 26 |
| IL6 (Forward) | 5′-GGAGACTTGCCTGGTGAAA-3′ | 27 |
| IL6 (Reverse) | 5′-CTGGCTTGTTCCTCACTACTC-3′ | 28 |
| IL8 (Forward) | 5′-CTTGGCAGCCTTCCTGATTT-3′ | 29 |
| IL8 (Reverse) | 5′-TTCTTTAGCACTCCTTGGCAAAA-3′ | 30 |
| TNFα (Forward) | 5′-CCAGGGACCTCTCTCTAATCA-3′ | 31 |
| TNFα (Reverse) | 5′-TCAGCTTGAGGGTTTGCTAC-3′ | 32 |
| TNFRSF11A | 5′-GAGTAGAACATCATGGGACAGAG-3′ | 33 |
| (Forward) | | |
| TNFRSF11A | 5′-ACGCGAAGAGAAGCAGAATTA-3′ | 34 |
| (Reverse) | | |
| IL1β (Forward) | 5′-ATGGACAAGCTGAGGAAGATG-3′ | 35 |
| IL1β (Reverse) | 5′-CCCATGTGTCGAAGAAGATAGG-3′ | 36 |
| PRL (Forward) | 5′-CTGTATCATCTGGTCACGGAAG-3′ | 37 |
| PRL (Reverse) | 5′-CGTTTGGTTTGCTCCTCAATC-3′ | 38 |
| ESR1 (Forward) | 5′-GTGCCAGGCTTTGTGGATTT-3′ | 39 |
| ESR1 (Reverse) | 5′-GGATCTCTAGCCAGGCACATTC-3′ | 40 |
| PGR (Forward) | 5′-AATGGAAGGGCAGCACAACTAC-3′ | 41 |
| PGR (Reverse) | 5′-TTTCTGCGGATTTTATCAACGAT-3′ | 42 |
| VEGFC (Forward) | 5′-AACAAGGAGCTGGATGAAGAG-3′ | 43 |
| VEGFC (Reverse) | 5′-CTGGCATGAGTTTCTGTCTAGT-3′ | 44 |
| PDGFA (Forward) | 5′-GGTGGCCAAGGTGGAATAC-3′ | 45 |
| PDGFA (Reverse) | 5′-CACTCCAAATGCTCCTCTAACC-3′ | 46 |
| HTR1B (Forward) | 5′-GTGGGTCTTCTCCATCTCTATCT-3′ | 47 |
| HTR1B (Reverse) | 5′-TAGAGGATGTGGTCGGTGTT-3′ | 48 |

### Immunoblotting.

Protein extracted from the xenografts underwent immunoblotting as previously described [25]. Primary antibodies targeting COL3A1, FN1, SPARC, TDO2, and Cleaved Caspase 3 were purchased from Proteintech Group, Inc. (Chicago, IL, USA). Band densities of specific proteins were quantified using the Image J program (http://imagej.nih.gov/ij/), and normalized to a Ponceau S staining band on the membrane. Results were expressed as means±SEM, and are presented as ratios relative to the control group, designated as 1.

### Enzyme-Linked Immunosorbent Assay.

The total collagen content in xenografts was assessed in duplicate using the QuickZyme Total Collagen Assay Kit (QuickZyme Biosciences, Leiden, Netherlands), following the manufacturer's instructions as previously described [20]. Absorbance readings were taken spectrophotometrically at a wavelength of 570 nm, and concentrations were determined by comparing sample optical density values to a standard curve. Total collagen levels were quantified as μg/ml of protein and reported as fold change relative to the vehicle group.

### Immunohistochemistry.

Tissues were fixed in 4% paraformaldehyde and subsequently embedded in paraffin, and 5-micron sections were cut and mounted onto glass slides. Staining procedures, including Masson's trichrome stain (HT15-1 KT, Sigma-Aldrich), and immunohistochemistry, were conducted as previously described [20, 22]. Primary antibodies utilized in this study included rabbit anti-Ki67 (dilution 1:250, 27309-1-AP, Proteintech Group, Inc, Rosemont, IL, USA), rabbit anti-cleaved caspase-3 (dilution 1:50, #9664, Cell Signaling Technology, Danvers, MA, USA), rabbit anti-CCND1 (dilution 1:50, #55506, Cell Signaling Technology), and mouse anti-E2F1 (dilution 1:50, sc-251, Santa Cruz Biotechnology, Inc., Dallas, TX, USA).

### Fibroid Explant Culture.

Equal weights of fibroid explants, aseptically cut from the same patient, were plated in 6-well plates containing complete medium. They were then incubated for 48 hours with either vehicle or Bay (5 μM).

### Statistics and Power Analysis.

The data was analyzed using GraphPad Prism 10 software (GraphPad, San Diego, CA, USA). Normality was assessed using the Kolmogorov-Smirnov test, and as the data were not normally distributed, non-parametric tests were employed for analysis. Wilcoxon matched pairs signed rank test was used for comparisons between two groups, while correlation analysis utilized the Spearman test. Statistical significance was established at p<0.05.

### III. Results

Bay was well-tolerated with no effects on the body weight of mice and no changes in blood chemistry including liver, kidney, and pancreatic function (Table 1).

TABLE 1
Chemistry panel results indicate no adverse
effects of in vivo Bay 11-7082 treatment

| | Vehicle | Bay 11-7082 | P- |
|---|---|---|---|
| Chemistry Panel Marker | (mean ± SEM) | (mean ± SEM) | value |
| General metabolism | | | |
| Glucose (mg/dl) | 230.9 ± 17.73 | 261.0 ± 14.63 | 0.069 |
| Kidney function | | | |
| BUN (mg/dl) | 24.3 ± 2.8 | 27.1 ± 5.75 | 0.648 |
| Creatinine (mg/dl) | 0.2 ± 0.29 | 0.4 ± 0.03 | 0.939 |
| Electrolytes | | | |
| Sodium (mEq/l) | 154.4 ± 0.98 | 155.3 ± 1.35 | 0.628 |
| Phosphorus (mg/dl) | 8.7 ± 0.77 | 10.1 ± 0.53 | 0.988 |
| Liver function | | | |
| Alkaline phosphatase (U/l) | 105 ± 16.95 | 91.9 ± 20.05 | 0.355 |
| Albumin (g/dl) | 3.9 ± 0.12 | 3.5 ± 0.14 | 0.063 |
| SGPT (ALT) (U/l) | 44 ± 10.3 | 54.5 ± 8.71 | 0.504 |
| Total protein (g/dl) | 5.2 ± 0.61 | 4.6 ± 0.16 | 0.326 |
| Globulin (g/dl) | 1.3 ± 0.5 | 1.1 ± 0.24 | 0.786 |
| Total bilirubin (mg/dl) | 0.2 ± 0.02 | 0.3 ± 0.04 | 0.732 |
| Pancreas function | | | |
| Amylase (U/l) | 703.9 ± 32.93 | 643.0 ± 36.82 | 0.074 |

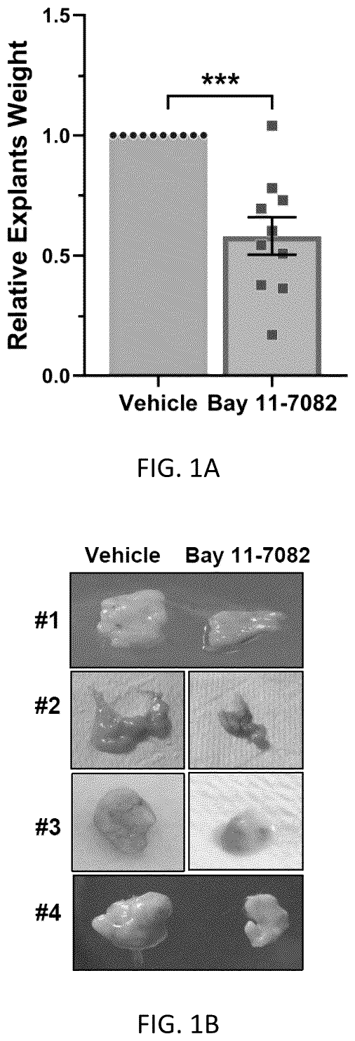

The administration of Bay led to 50% reduction of tumor weight after two months of treatment (FIG. 1A and FIG. 1B).

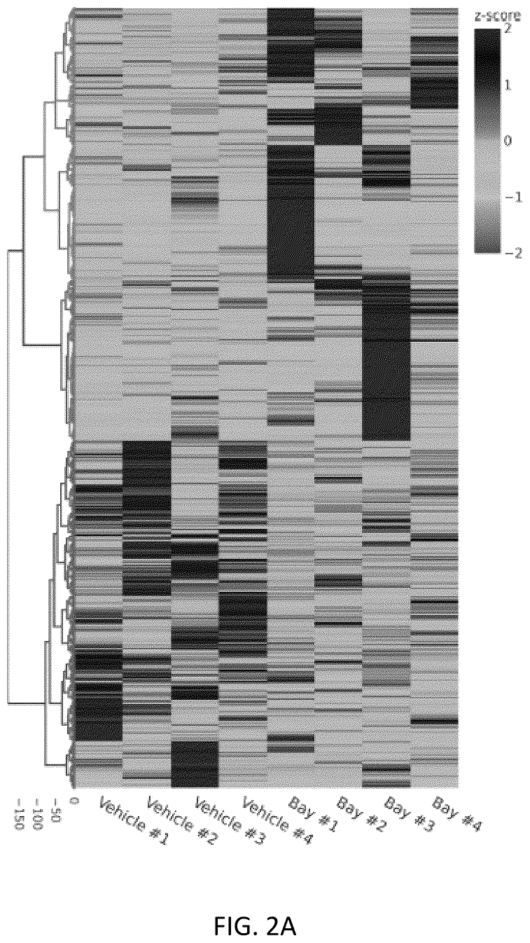

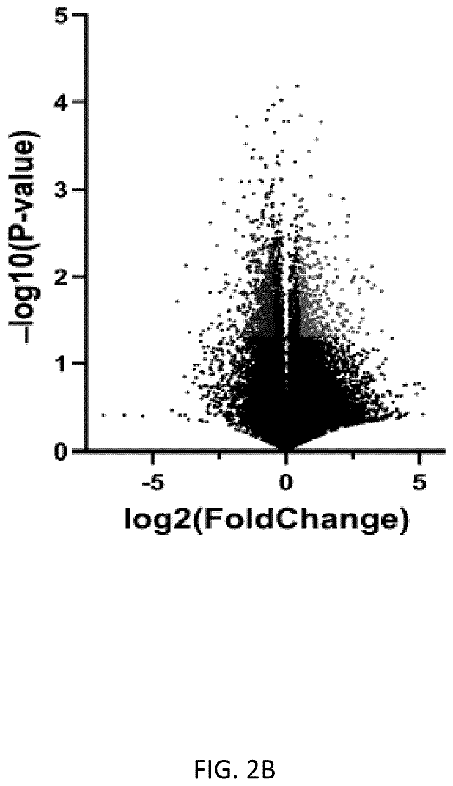

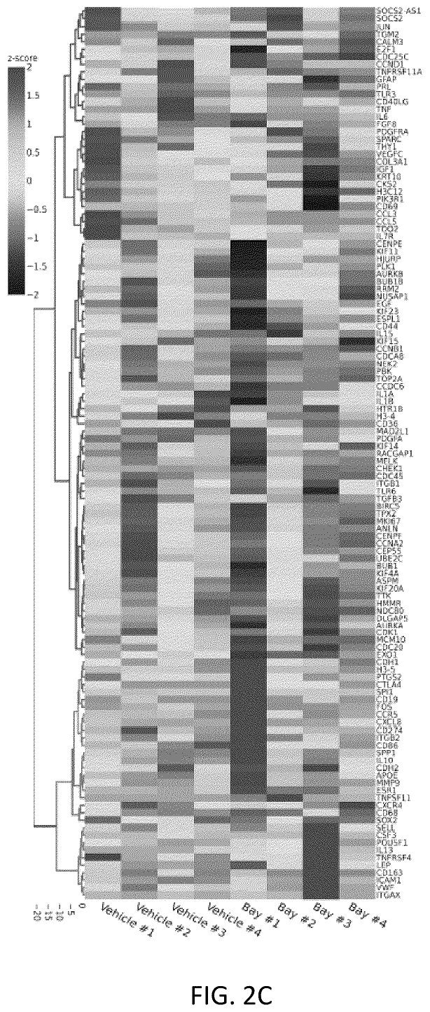

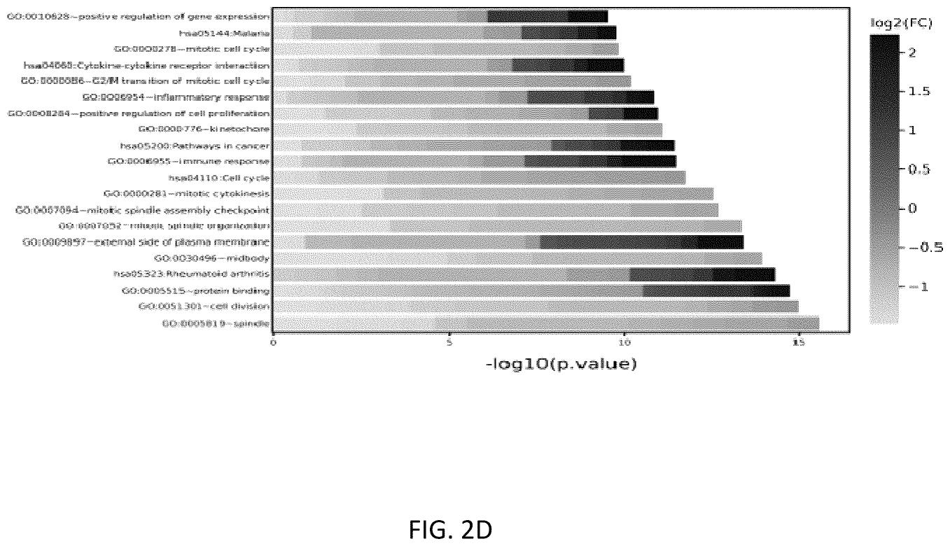

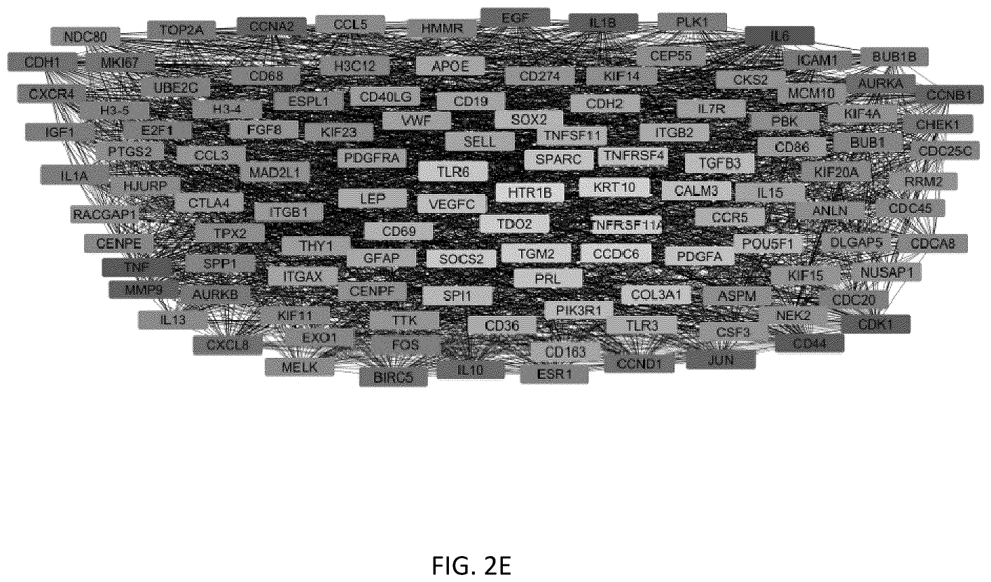

The results of RNAseq on the xenografts from 8 mice (4 treated with vehicle and 4 with Bay) after two months of treatment are shown in FIGS. 2A-2E. Xenografts from the same patient were exposed to either vehicle control or Bay thus allowing a matched comparison. As shown in the heat map (FIG. 2A) and volcano plot (FIG. 2B), Bay altered the expression of numerous genes mostly inhibiting their expression. Using a cutoff of 1.5 fold, 374 transcripts were downregulated and 629 transcripts were upregulated. The heat map (FIG. 2C) shows 119 enriched hub transcripts identified by the CytoHubba plugin of the Cytoscape software platform. KEGG and Gene ontology (GO) enrichment analysis (FIG. 2D) indicated that the differentially expressed genes primarily fell in the categories of cell proliferation and processes related to it such as mitotic spindle organization, and kinetochore; inflammatory and immune response and cytokine-cytokine receptor interaction. Network analysis indicated the complex potential interaction among the altered genes (FIG. 2E). As shown in FIG. 2C, the vast majority of genes were downregulated and were primarily involved with cell proliferation, mitosis, mitotic apparatus, and cytokinesis (e.g., Ki67, E2F1, CCND1, CCNB1, CCNA2, BUB1, CKS2 and others). The drug induced a decrease in the expression of a number of growth factors known to be upregulated in fibroid (e.g IGF1, EGF, PDGF, FGF8, PRL, TGFB3), and several components of the ECM such as COL3A1, KRT10. The drug also induced downregulation of several pro-inflammatory cytokines known to be overexpressed in fibroids including IL1 a and 1 p, TNF, IL6, IL15, IL7R, TNFRSF11A aka RANK, and downregulation of TLR3 and TLR6 whose function is activation of NF-kB, and a number chemokines (CXCCR4, CCR5, CCL3, CCL5). In addition, Bay significantly inhibited the expression of TDO2 which was recently reported to be highly overexpressed in fibroids [26-28], and related to the tryptophan catabolic pathway, the serotonin receptor, HTR1 B. There was significant variation among the animals in the up-regulated genes with a lack of consistency among the xenografts but notable genes were vWF, ICAM1, ITGAX, SOX2, CXCL8, IL10, and IL13.

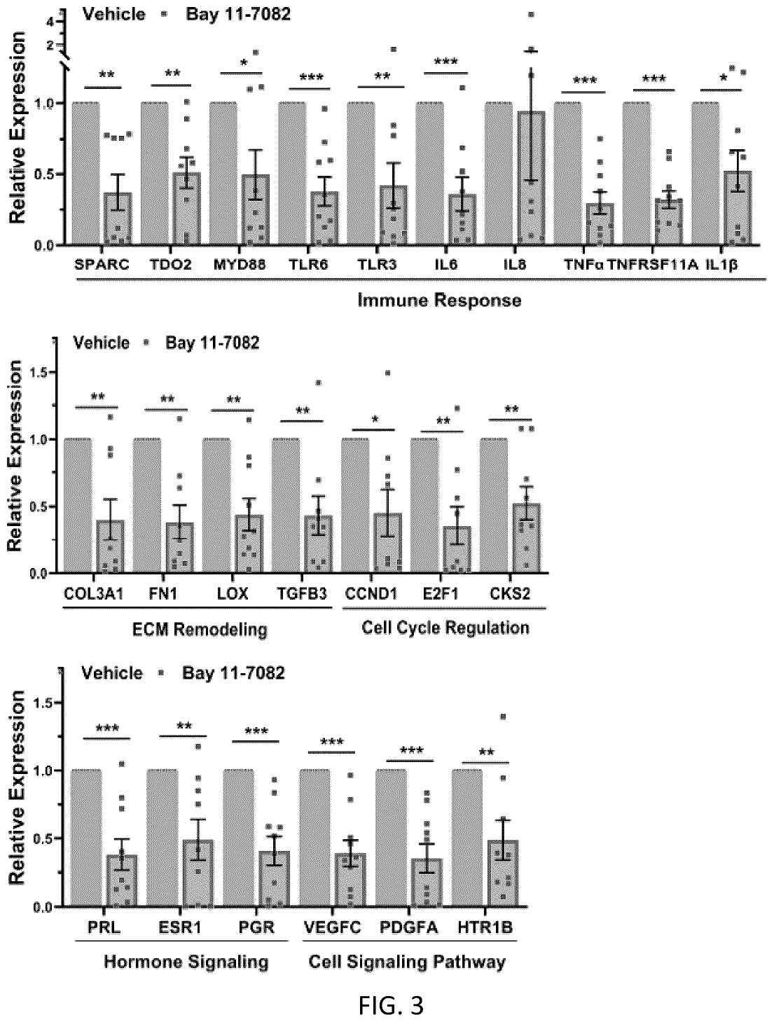

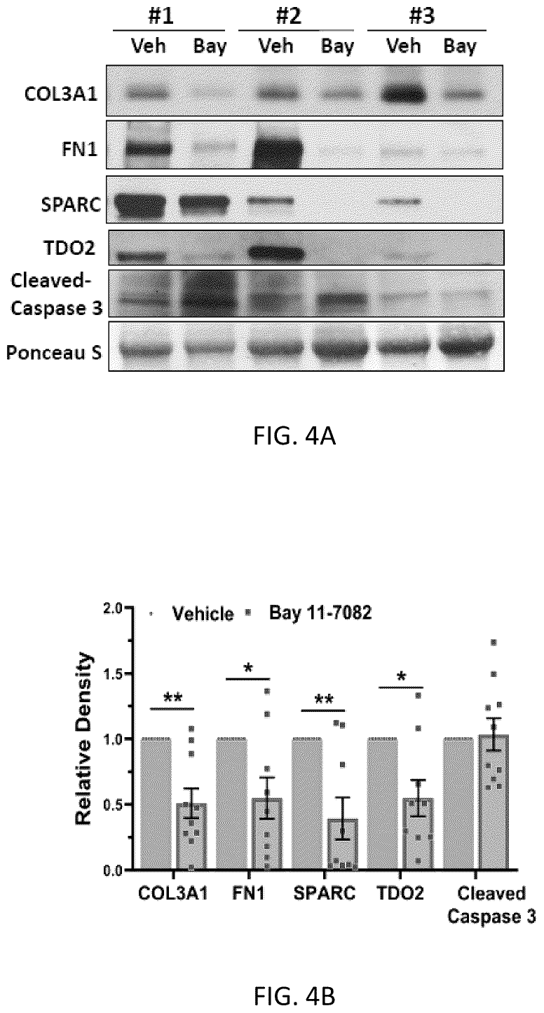

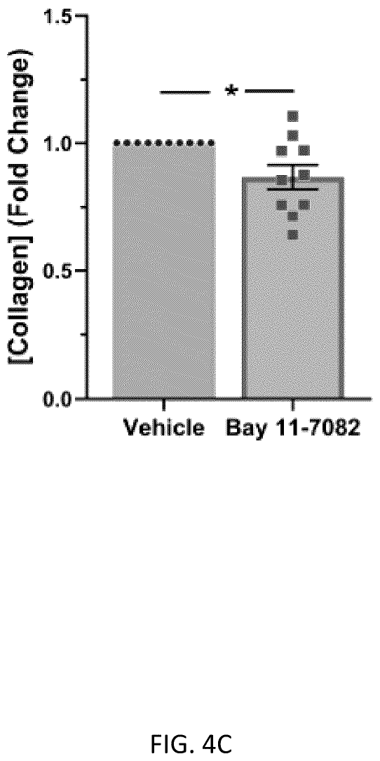

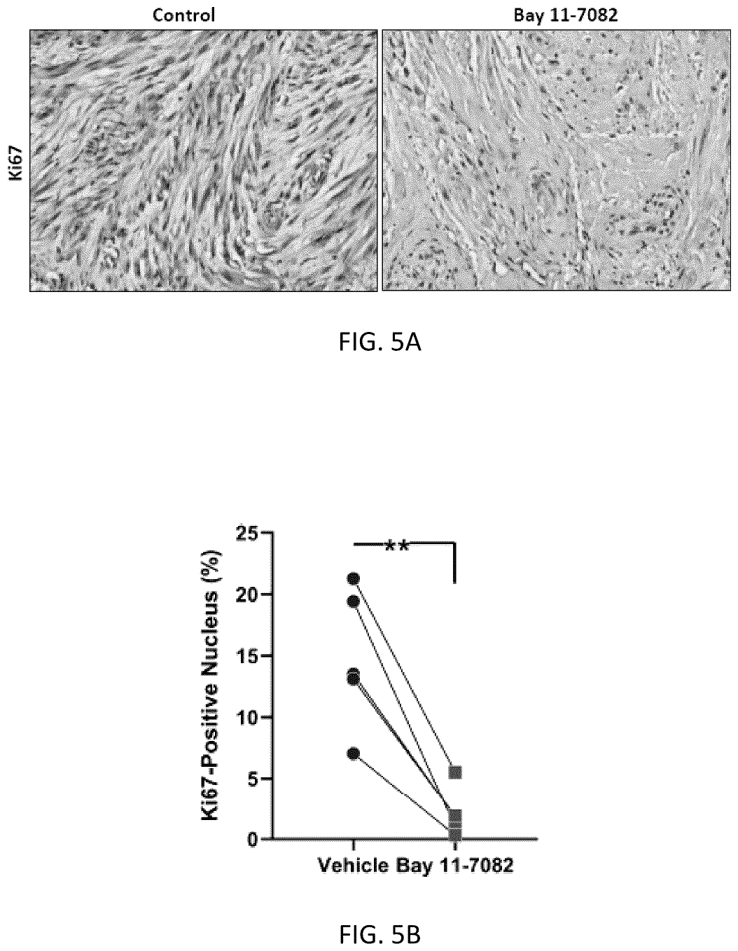

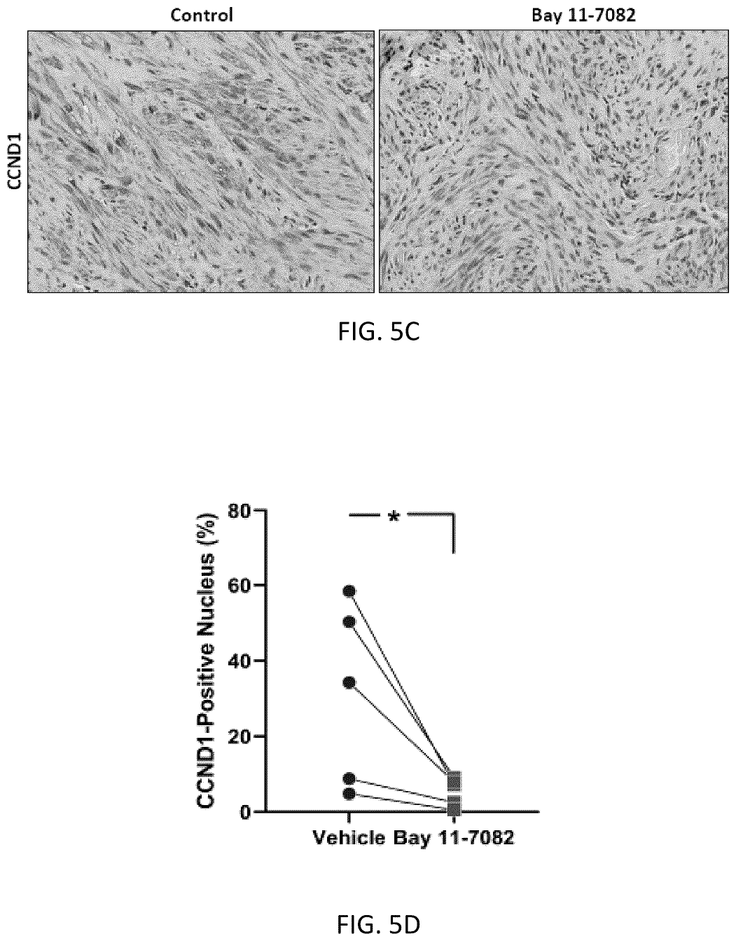

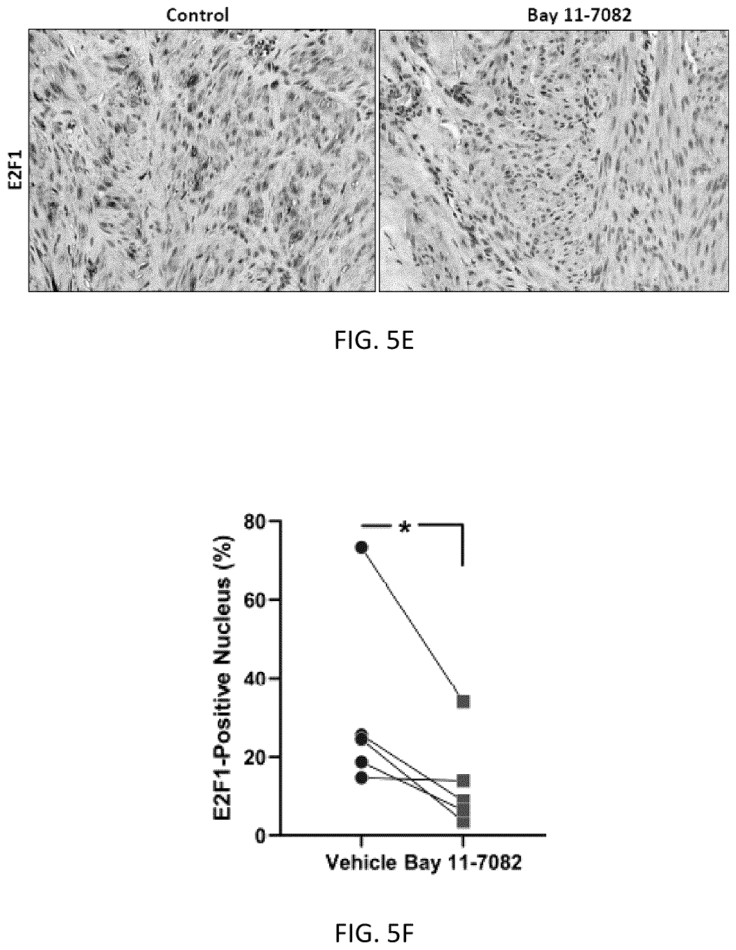

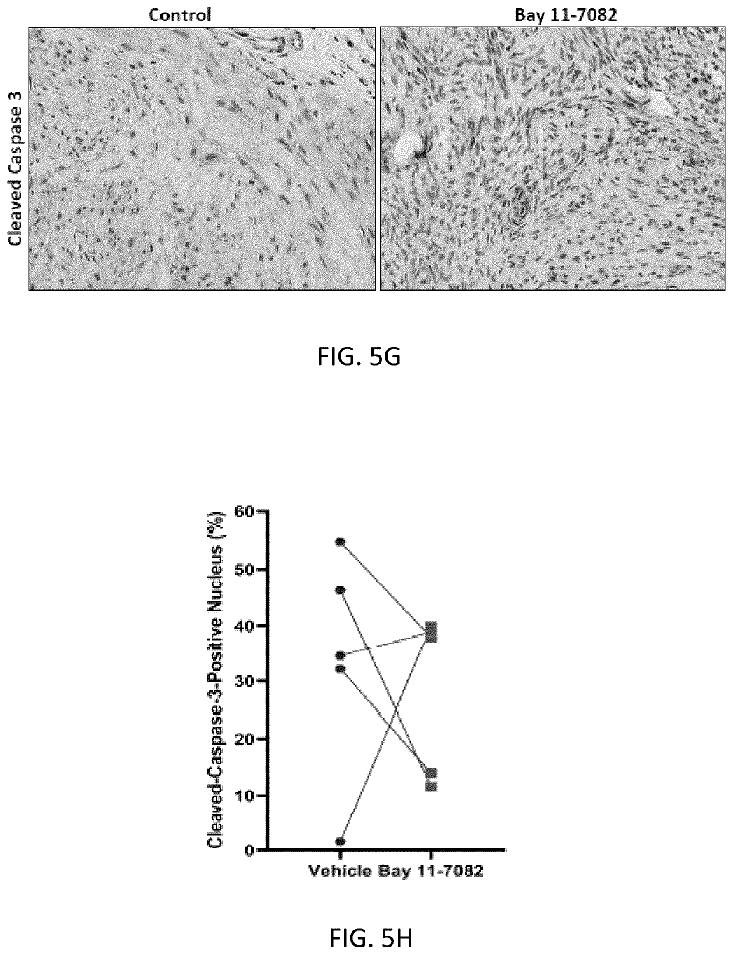

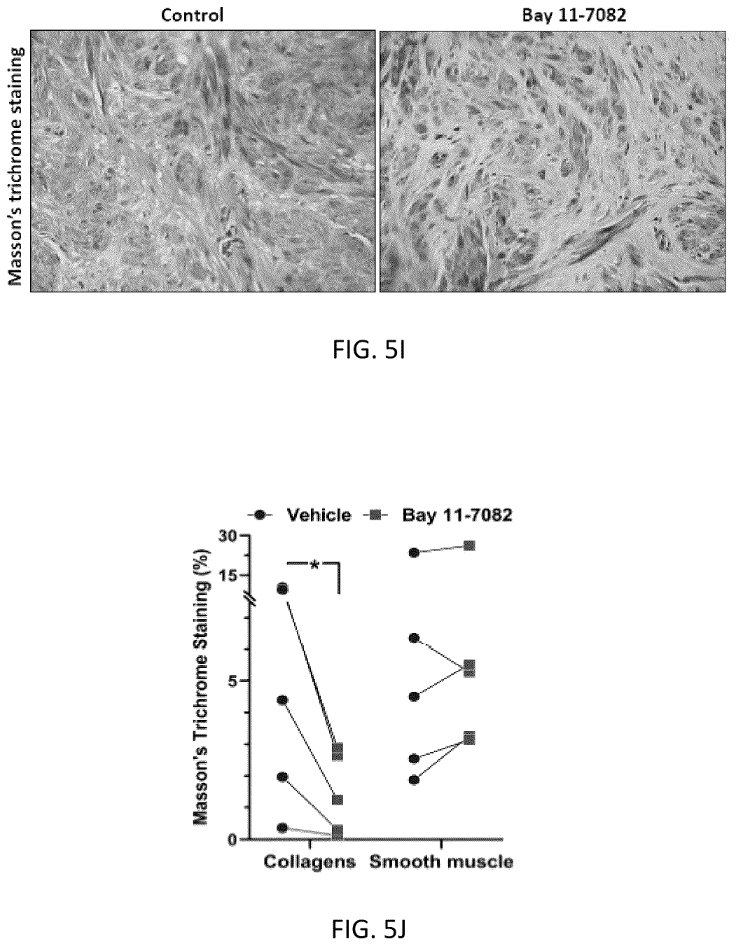

Based on the RNAseq data, validation studies using qRT-PCR (FIG. 3), Western blot analysis (FIGS. 4A-4C), and IHC (FIGS. 5A-5J) were performed for select genes. As shown in FIG. 3, Bay induced a significant decrease in the mRNA expression of genes involved in inflammatory response (SPARC, TDO2, MYD88, TLR6, TLR3, IL6, TNFα, TNFRSF11A, IL1β but not IL8), ECM remodeling (COL3A1, FN1, LOX, TGFB3), cell cycle regulation (CCND1, E2F1, CKS2), genes involved in hormone signaling (PRL, ESR1, PGR), and cell signaling (VEGFC, PDGFA, HTR1 B). Due to limited sample availability, Western blot analysis was only performed for a limited number of genes shown in FIG. 4A and FIG. 4B. As demonstrated in FIG. 4B, Bay inhibited the protein abundance of COL3A1, FN1, SPARC, and TDO2 but not cleaved Caspase 3. Total collagen protein levels were also measured in the xenografts (FIG. 4C) and a significant decrease in total collagen protein levels was shown in the xenografts of Bay treated animals as compared with controls. Further confirmation studies were performed by IHC followed by Image analysis using the Halo software in sections of xenografts (FIGS. 5A-5J). There was a significant reduction in the expression of the proliferation marker Ki67 in the drug-treated mice (FIG. 5A and FIG. 5B), and decreased expression of several cell cycle markers including CCND1 (FIG. 5C and FIG. 5D), and E2F1 (FIG. 5E and FIG. 5F), which could account for the reduction in tumor weight in response to Bay. In line with western blot analysis, no significant difference in Cleaved caspase 3 staining in drug versus vehicle-treated controls was detected (FIG. 5G and FIG. 5H). Masson's trichrome staining of xenograft sections revealed a significant decrease in the expression of collagens in Bay-treated xenografts and no significant differences in smooth muscle content in the two groups (FIG. 5I and FIG. 5J).

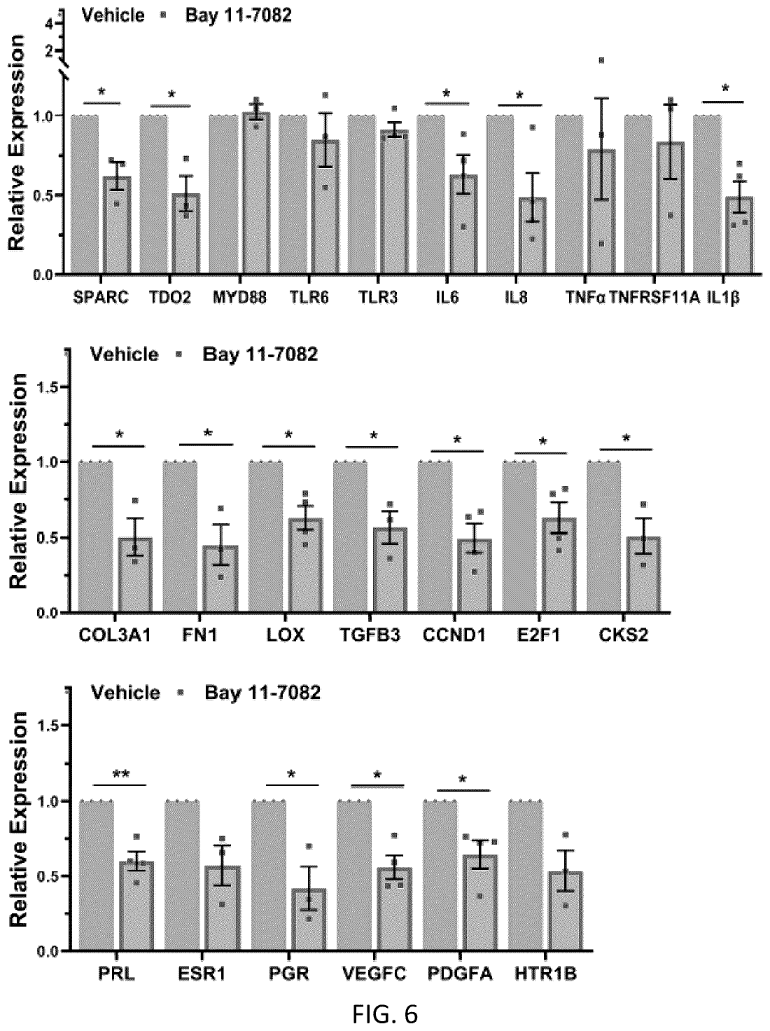

In vitro experiment was performed to determine which of the observed in vivo effects of Bay on gene expression are due to the direct effect of Bay on gene transcription. As shown in FIG. 6, treatment of human fibroid explants with Bay (5 μM) after 48 hours induced a significant inhibition of VEGFC, TDO2, TGFB3, LOX, E2F1, PRL, PGR, CKS2, SPARC, COL3A1, FN1, CCND1, IL8, IL6 and IL1p mRNA levels without significant effects on TLR3 and 6, MyD88, TNFα, ESR1, HTR1 B, and TNFRSF11A.

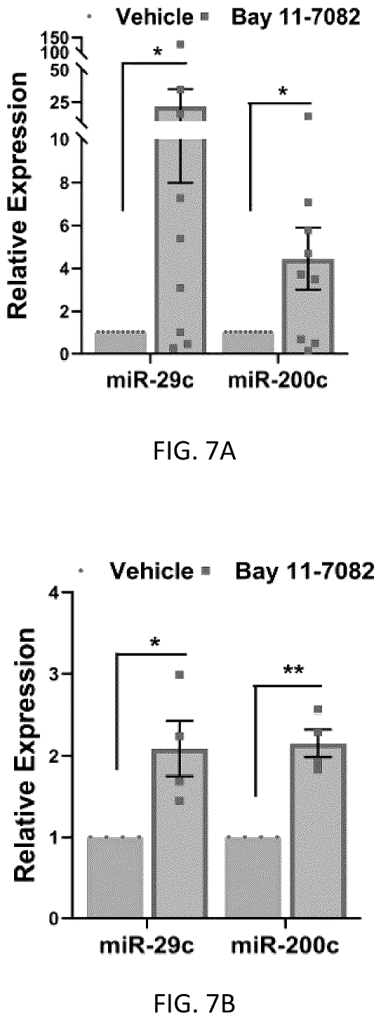

Based on prior studies indicating an interaction between NF-kB and miR-29c and miR-200c both of which are downregulated in fibroids [10, 11], the expression of these miRNAs was quantified in the fibroid xenografts (FIG. 7A) and human fibroid explants (FIG. 7B). As shown in FIG. 7A and FIG. 7B, treatment with Bay led to a significant increase in the expression of miR-29c and miR-200c in the xenografts and in fibroid explants.

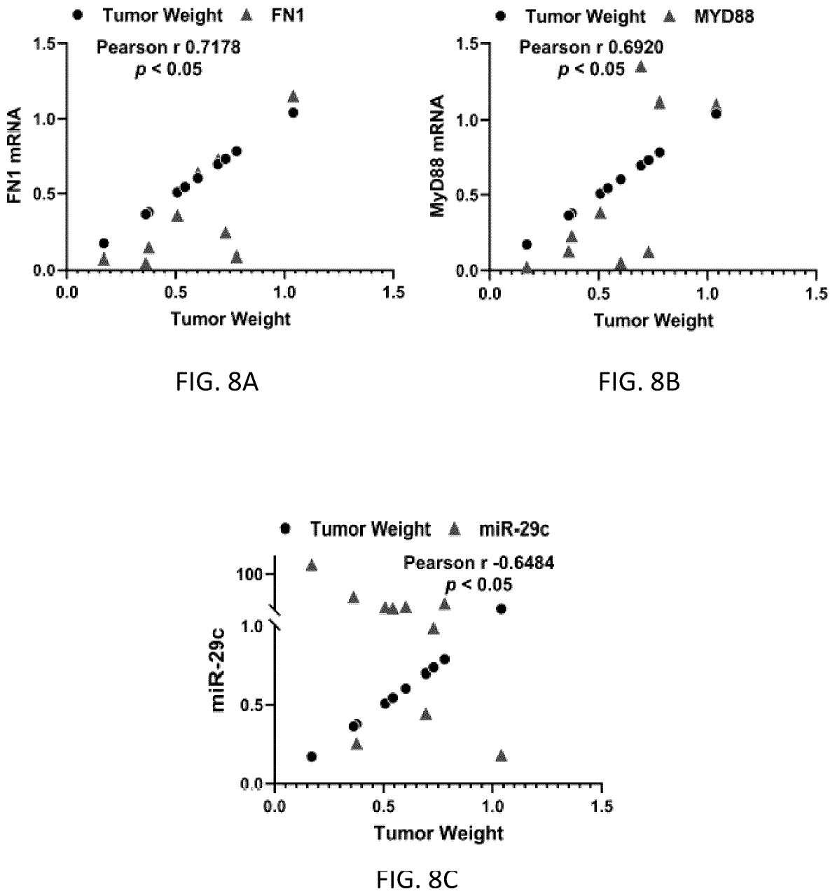

A correlation analysis was performed between fibroid weight and the expression of various genes shown in FIG. 3 and FIG. 6. As demonstrated in FIGS. 8A-8C, there was a significant positive correlation between tumor weight after two months of treatment with Bay or vehicle and the expression of FN1, a major component of the ECM [29], the inflammatory transcription factor MyD88 [30] and miR-29c, a major regulator of ECM genes [10] but not any other genes shown in FIG. 3 and FIG. 6.

### IV. Discussion

The results of this study indicate that in vivo administration of Bay, a NF-kB inhibitor, is well tolerated and highly effective in inhibiting the growth of fibroids, decreasing tumor weight by 50% after two months of treatment. This response to the drug was independent of the MED12 mutation status of the fibroid or the race/ethnicity of the patients. This decrease in tumor weight is primarily due to Bay's effects inhibiting the expression of genes regulating the cell cycle and cell proliferation including CCND1, E2F1, CKS2, and Ki67. Bay also significantly reduced ECM accumulation a hallmark of uterine fibroids by inhibiting the expression of collagens, including COL3A1, FN1, and LOX. The drug was also effective in inhibiting the expression of a number of genes involved in the immune response and inflammation (SPARC, TDO2, MyD88, TLR3 and 6, IL6, TNFα, TNFRFSF11A, IL1β) in the fibroid xenografts. Bay significantly decreased the expression of ESR1/PGR which are critical for the growth and progression of fibroids [31, 32], and a number of growth factors known to be upregulated in fibroids including prolactin [33, 34], VEGF [35, 36], PDGFA [37]. The effectiveness of Bay to shrink fibroid tumors by 50% and its induction of a favorable gene profile to inhibit genes associated with the cell cycle, ECM, and inflammation makes this and other drugs with similar modes of action a promising novel therapy for fibroids.

There is an established connection between inflammation and tumorigenesis as such aberrant activation of NF-kB has been reported in multiple cancers [38]. Activation of NF-kB results in upregulation of genes regulating cell proliferation and migration and downregulation of genes regulating apoptosis [38-40]. This transcription factor also plays a key role in regulating the expression of genes in epithelial-mesenchymal transition [41] and cancer cell stemness [42]. Because of the role of NF-kB in oncogenesis, there has been significant interest in targeting it for cancer therapy [39, 41, 43]. Fibroid tumors are characterized by increased cell proliferation and increased expression of cell cycle proteins [44]. RNAseq data indicated that in vivo administration of Bay had the greatest effect on genes associated with cell cycle and cell proliferation in the fibroid xenografts and confirmatory analysis showed decreased expression of a number of these genes including CCND1 (cyclin D1) which regulates G1/S transition and is overexpressed in a variety of tumors [45], E2F1 a transcription factor and cell cycle regulator which can mediate both cell proliferation and apoptosis [46], and CKS2 which binds to cyclin-dependent kinases and is essential for their function [47].

Previous reports in various types of cancer have shown the induction of apoptosis by Bay [15, 16, 49, 50]. However, in vivo studies show Bay did not induce apoptosis in the fibroid xenografts as determined by cleaved caspase 3 protein levels and IHC analysis, although there were significant variations among the xenografts with some exhibiting increased apoptosis. This unexpected finding could be secondary to the drug dose used, which may not have been high enough to induce apoptosis and the limited sample number of animals.

A hallmark of fibroids is excess accumulation of ECM [1]. The data indicates that Bay reduced the expression of the main components of ECM, namely collagen and fibronectin. The reduction in tumor weight could be secondary to decreased ECM accumulation as reflected by the positive correlation between tumor weight and FN1 levels. A critical regulator of fibrosis is the TGF-β cytokine family which is produced by diverse cells and with critical roles in immunity [51], fibrosis [52], and cancer [53]. TGFβ3 is upregulated in fibroids and is critical in the overproduction of ECM in fibroids [54-57]. Bay significantly reduced the expression of TGFβ3 in the fibroid xenografts which could be one of the mechanisms for the reduction of collagen and FN1 levels. Several studies have indicated a cross-talk between the TGF-β and NF-kB pathways [58-60]. TGF-β/SMAD was shown to be regulated by the interaction of SMAD3 with IKKα in breast cancer cells [61]. Similarly, TGF-β was shown to activate NF-kB by TGF-p activated kinase 1. This results in the phosphorylation of IKBα leading to proteasomal degradation of IKBα and the release of NF-kB p65/p50 heterodimer and NF-kB activation [62]. Another gene that was validated and related to ECM remodeling was Lysyl Oxidase (LOX) which was inhibited by Bay. LOX is a copper-dependent amine oxidase that catalyzes the cross-linking of collagen and elastin thus contributing to ECM stiffness [63]. LOX not only plays a role in physiologic processes like organ development [64], but also in pathologic processes such as tumorigenesis [65, 66] and progression of fibrosis [67]. The induction of NF-kB by advanced glycation end products was shown to upregulate lysyl oxidase expression in endothelial cells [68]. LOX also interacts with a number of growth factor signaling pathways identified in this study including VEGF, PDGF, and TGF-3 [69]. To date, there are no reports on the effect of Bay on LOX expression.

The growth of fibroids is dependent on the stimulatory effects of estrogen and progesterone [1]. The inhibition of estrogen and progesterone receptor expression by Bay provides a significant beneficial effect of this drug for tumor growth, making them less responsive to the mitogenic effect of these ovarian steroids. Previous studies have shown a direct negative interaction between NF-kB and estrogen, progesterone, and glucocorticoid receptors [72]. Furthermore, both progesterone and glucocorticoid activated IKBα promoter thereby inhibiting NF-kB in breast cancer cells [73], and estradiol-induced estrogen response element activity in endometrial cells by an NF-kB dependent and estrogen receptor mechanism [74]. The in vivo effect of Bay on estrogen/progesterone receptor expression has not been reported to date. Bay also inhibited the expression of PRL in the xenografts. Several studies have indicated that PRL is expressed and secreted by fibroid and myometrial tissue and that it stimulates cell proliferation [34, 75]. Thus, reduction of PRL expression by Bay is another beneficial effect of this drug to reduce fibroid growth and ECM volume. Fibroids are also characterized by increased angiogenesis with increased microvessel density and VEGF expression [76]. Bay effectively inhibited VEGFC mRNA expression in fibroid xenografts. Other studies have shown the importance of NF-kB in tumor angiogenesis and the role of VEGF in this process [76]. PDGF is another growth factor that promotes cell proliferation and was previously reported to be upregulated in fibroids [77]. In vivo administration of Bay significantly reduced the expression of PDGF. Previous studies showed the role of NF-kB in regulating PDGF expression in fibroblasts [78]. Little is known about serotonin and its receptors in fibroids. Collectively the inhibition of expression of the growth factors such as PRL, PDGF, HTR1 B, and VEGF in fibroid xenografts provides another mechanism for reducing tumor cell proliferation and angiogenesis in fibroids.

The inhibition of canonical NF-kB by Bay expectedly inhibited the expression of pro-inflammatory cytokines IL6, IL1β, and TNFα. These cytokines are also activators of canonical NF-kB [80] and among them, IL1p and TNFα are overexpressed in fibroids [6, 81]. Bay also inhibited several genes upstream of the NF-kB pathway including TLRs and MyD88. Toll-like receptors are pattern recognition receptors that through adaptor proteins such as MyD88 convey messages to the NF-kB complex. TLR6 is a membrane-associated receptor whereas TLR3 is endosome-associated [82, 83]. Activation of TLR6 receptors results in its interaction with MyD88, and activation of TLR3 with the protein TRIF both of which lead to the activation of NF-kB [84]. There is only one report indicating activation of NF-kB and TLR4 in fibroid cells and uterine fibroid-derived fibroblast [85] and no published reports on MyD88 in fibroids. Thus, Bay not only inhibits NF-kB but also upstream inputs that activate the canonical NF-kB including pro-inflammatory cytokines TNFα and IL1β, toll-like receptors, and their adaptor proteins through yet unidentified mechanisms.

In vivo treatment with Bay also inhibited SPARC a matricellular protein that plays a key role in tissue repair and wound healing and has a wide range of functions in ECM production, inflammation, cell cycle, growth factor activity, and cell adhesion [86]. SPARC is overexpressed in inflammatory conditions such as rheumatoid arthritis and different types of cancer [86]. A previous report showed a marked overexpression of SPARC mRNA which was dependent on the MED12 mutation status of fibroid [87]. In bone, NF-kB activation was shown to stimulate SPARC [88] and SPARC inhibited the metabolic programming of ovarian cancer cells and adipocytes by an NF-kB-mediated mechanism [89]. Dysregulation of Tryptophan catabolism has been shown in fibroids, with a marked overexpression of TDO2 which also was race and MED12 dependent [26, 27], and its inhibition in vitro [26] and in vivo [28] resulted in decreased fibroid cell proliferation, decreased ECM accumulation, and reduced inflammation in fibroids. The data supports the existence of this cross-talk, showing inhibition of NF-kB by Bay reduces the expression of TDO2 which would contribute to the inhibition of fibroid cell proliferation, ECM production, and inflammation.

In this study, the expression of two key miRNAs in fibroid pathogenesis were measured, both of which are downregulated in fibroids namely miR-29c which regulates many components of the ECM such as collagens, and miR-200c which regulates many of the cell cycle and inflammation regulatory proteins [1]. It has been shown that treatment with Bay and silencing p65 by siRNA both induced the expression of miR-29c and miR-200c in primary fibroid smooth muscle cells [10, 93], indicating the significant involvement of NF-kB in their regulation. It has been shown that treatment with Tranilast reduces p65 nuclear translocation, thereby diminishing its binding capacity to the miR-200c promoter, leading to the induction of miR-200c [93]. Other studies have shown that miR-29c is epigenetically silenced by an activated NF-κB/YY1 complex, which functions as a suppressor of the miR-29c promoter in Rhabdomyosarcoma (RMS) [94]. Similarly, miR-200c inhibits IL8 in fibroids through an NF-kB-mediated mechanism [11]. In this study, it is shown that treatment of fibroid xenografts and human fibroid explants with Bay significantly increases the expression of both miR-29c and miR-200c. The overexpression of these miRNAs would be expected to result in decreased expression of their target genes including those associated with cell cycle regulation, ECM, and inflammation. The lack of change in IL8 a target of miR-200c in this study was unexpected but could be due to secondary effects of Bay or inadequate dose of drug.

In vitro experiments with fibroid explants which preserve all the cell types were used to determine which of the in vivo effects of Bay on gene expression are due to direct effects of NF-kB on gene transcription. With the exception of TLR3 and 6, MyD88, and TNFRSF11A all the observed inhibitory effects of Bay in the in vivo study were shown to be at least in part due to a direct involvement of NF-kB in their transcription. Epigenetic mechanisms such as alteration of expression of some miRNAs as shown for miR-29c and miR-200c, or altered expression of growth factors could be other mechanisms for the indirect effects of NF-kB inhibitor on gene transcription.

Various drugs that target the complex NF-kB pathway at different levels of the pathway are in development for the treatment of inflammatory conditions such as rheumatoid arthritis, autoimmune diseases, and cancer, and some of these drugs have already gained FDA approval [7, 84]. This study indicates the usefulness of inhibiting the canonical NF-kB pathway by Bay for the treatment of fibroids. In a previous in vivo study it was shown that Tranilast which is approved for the treatment of asthma and fibrotic diseases in Asia also has beneficial effects for the treatment of fibroids by reducing tumor size and inhibiting collagen expression and cell proliferation and increasing cellular apoptosis [22]. In vitro studies indicated that Tranilast works by inducing the expression of miR-200c which in turn inhibited the NF-KB signaling pathway by preventing nuclear translocation of ReIA-p65 [93]. The non-canonical NF-kB could also be targeted for fibroid treatment as shown in a previous study where its inhibition by RANK-Fc in vivo blocked RANKL-induced expression of cyclin D1, decreasing fibroid growth and expression of Ki67 [95]. Inhibition of the canonical NF-kB pathway by Bay also indirectly inhibits TNFRSF11A which is upregulated in fibroid stem cells [95].

In summary, this preclinical study indicates that in vivo administration of Bay is well tolerated without side effects. The inhibition of the canonical NF-kB pathway has a beneficial effect in reducing the growth and progression of human fibroids in a mouse xenograft model. This reduction in tumor weight is accompanied by the induction of a highly favorable gene expression profile in the fibroid xenografts with reduced expression of cell cycle regulatory proteins, components of the ECM such as collagen and FN1, pro-inflammatory cytokines (IL6, IL1β, TNFα), estrogen and progesterone receptor, growth, and angiogenic factors (PDGF, VEGFC, SPARC and TDO2 and induction of two miRNAs with critical roles in fibroid pathogenesis namely miR-29c and miR-200c. Bay also inhibited genes upstream of NF-kB signaling including TLR 3 and 6 and MyD88 and the non-canonical activator TNFRFSF11 A (RANK). In vitro experiments with Bay in fibroid explants indicated that the in vivo inhibitory effects of Bay on these genes were probably due to direct inhibitory effects of NF-kB, with the exception of TLR3 and 6, MyD88 and TNFRSF11A. These promising data warrant further exploration of the NF-kB pathway for fibroid treatment, and potentially repurposing some already approved drugs.

### V. References

1. Baranov, V. S.; Osinovskaya, N. S.; Yarmolinskaya, M. I. Pathogenomics of Uterine Fibroids Development. Int J Mol Sci 2019, 20, (24).
2. Reis, F. M.; Bloise, E.; Ortiga-Carvalho, T. M. Hormones and pathogenesis of uterine fibroids. Best Pract Res Clin Obstet Gynaecol 2016, 34, 13-24.
3. Chegini, N. Proinflammatory and profibrotic mediators: principal effectors of leiomyoma development as a fibrotic disorder. Semin Reprod Med 2010, 28, (3), 180-203.
4. Konenkov, V. I.; Koroleva, E. G.; Orlov, N. B.; Prokof'ev, V. F.; Shevchenko, A. V.; Novikov, A. M.; Dergacheva, T. I. Blood Serum Levels of Proinflammatory Cytokines (IL-1β, IL-6, TNFα, IL-8, IL-12p70, and IFNy) in Patients with Uterine Myoma. Bull Exp Biol Med 2018, 165, (5), 698-701.
5. Orciani, M.; Caffarini, M.; Biagini, A.; Lucarini, G.; Delli Carpini, G.; Berretta, A.; Di Primio, R.; Ciavattini, A. Chronic Inflammation May Enhance Leiomyoma Development by the Involvement of Progenitor Cells. Stem Cells Int 2018, 2018, 1716246.
6. Zannotti, A.; Greco, S.; Pellegrino, P.; Giantomassi, F.; Delli Carpini, G.; Goteri, G.; Ciavattini, A.; Ciarmela, P. Macrophages and Immune Responses in Uterine Fibroids. Cells 2021, 10, (5).
7. Ramadass, V.; Vaiyapuri, T.; Tergaonkar, V. Small Molecule NF-KB Pathway Inhibitors in Clinic. Int J Mol Sci 2020, 21, (14).
8. Liu, T.; Zhang, L.; Joo, D.; Sun, S. C. NF-KB signaling in inflammation. Signal Transduct Target Ther 2017, 2, 17023-.
9. Taniguchi, K.; Karin, M. NF-KB, inflammation, immunity and cancer: coming of age. Nat Rev Immunol 2018, 18, (5), 309-324.
10. Chuang, T. D.; Khorram, O. Mechanisms underlying aberrant expression of miR-29c in uterine leiomyoma. Fertil Steril 2016, 105, (1), 236-245.e1.
11. Chuang, T. D.; Khorram, O. miR-200c Regulates IL8 Expression by Targeting IKBKB: A Potential Mediator of Inflammation in Leiomyoma Pathogenesis. PLoS One 2014, 9, (4), e95370.
12. AlAshqar, A.; Reschke, L.; Kirschen, G. W.; Borahay, M. A. Role of inflammation in benign gynecologic disorders: from pathogenesis to novel therapiest. Biol Reprod 2021, 105, (1), 7-31.
13. Zahid, A.; Li, B.; Kombe, A. J. K.; Jin, T.; Tao, J. Pharmacological Inhibitors of the NLRP3 Inflammasome. Front Immunol 2019, 10, 2538.
14. Lee, J.; Rhee, M. H.; Kim, E.; Cho, J. Y. BAY 11-7082 is a broad-spectrum inhibitor with anti-inflammatory activity against multiple targets. Mediators Inflamm 2012, 2012, 416036.
15. Wang, Y.; Zhang, X. L.; Sun, C. M. BAY-11-7082 induces apoptosis of multiple myeloma U266 cells through inhibiting NF-KB pathway. Eur Rev Med Pharmacol Sci 2018, 22, (9), 2564-2571.
16. Garcia, M. G.; Alaniz, L.; Lopes, E. C.; Blanco, G.; Hajos, S. E.; Alvarez, E. Inhibition of NF-kappaB activity by BAY 11-7082 increases apoptosis in multidrug resistant leukemic T-cell lines. Leuk Res 2005, 29, (12), 1425-34.
17. Chuang, T. D.; Ton, N.; Rysling, S.; Quintanilla, D.; Boos, D.; Gao, J.; McSwiggin, H.; Yan, W.; Khorram, O. The Influence of Race/Ethnicity on the Transcriptomic Landscape of Uterine Fibroids. Int J Mol Sci 2023, 24, (17), 13441.
18. Chuang, T. D.; Gao, J.; Quintanilla, D.; McSwiggin, H.; Boos, D.; Yan, W.; Khorram, O. Differential Expression of MED12-Associated Coding RNA Transcripts in Uterine Leiomyomas. Int J Mol Sci 2023, 24, (4), 3742.
19. Chuang, T. D.; Ton, N.; Rysling, S.; Boos, D.; Khorram, O. The Effect of Race/Ethnicity and MED12 Mutation on the Expression of Long Non-Coding RNAs in Uterine Leiomyoma and Myometrium. Int J Mol Sci 2024, 25, (2).
20. Chuang, T. D.; Ton, N.; Rysling, S.; Quintanilla, D.; Boos, D.; Khorram, O. Therapeutic Effects of In Vivo Administration of An Inhibitor of Tryptophan 2,3-dioxygenase (680C91) for the Treatment of Fibroids: A Preclinical Study. Fertil Steril 2023.
21. Dewan, M. Z.; Terashima, K.; Taruishi, M.; Hasegawa, H.; Ito, M.; Tanaka, Y.; Mori, N.; Sata, T.; Koyanagi, Y.; Maeda, M.; Kubuki, Y.; Okayama, A.; Fujii, M.; Yamamoto, N. Rapid tumor formation of human T-cell leukemia virus type 1-infected cell lines in novel NOD-SCID/gammac(null) mice: suppression by an inhibitor against NF-kappaB. J Virol 2003, 77, (9), 5286-94.
22. Chuang, T. D.; Munoz, L.; Quintanilla, D.; Boos, D.; Khorram, O. Therapeutic Effects of Long-Term Administration of Tranilast in an Animal Model for the Treatment of Fibroids. Int J Mol Sci 2023, 24, (13), 10465.
23. Iqbal, A., Duitama, C., Metge, F., Rosskopp, D., Boucas, J. Flaski. Flaski (3.12.2). Zenodo 2021.
24. Szklarczyk, D.; Franceschini, A.; Wyder, S.; Forslund, K.; Heller, D.; Huerta-Cepas, J.; Simonovic, M.; Roth, A.; Santos, A.; Tsafou, K. P.; Kuhn, M.; Bork, P.; Jensen, L. J.; von Mering, C. STRING v10: protein-protein interaction networks, integrated over the tree of life. Nucleic Acids Res 2015, 43, (Database issue), D447-52.
25. Chuang, T. D.; Quintanilla, D.; Boos, D.; Khorram, O. Long Noncoding RNA MIAT Modulates the Extracellular Matrix Deposition in Leiomyomas by Sponging MiR-29 Family. Endocrinology 2021, 162, (11), bqab186.
26. Chuang, T. D.; Quintanilla, D.; Boos, D.; Khorram, O. Tryptophan catabolism is dysregulated in leiomyomas. Fertil Steril 2021, 116, (4), 1160-1171.
27. Chuang, T. D.; Quintanilla, D.; Boos, D.; Khorram, O. Further characterization of tryptophan metabolism and its dysregulation in fibroids. F S Sci 2022, 3, (4), 392-400.
28. Chuang, T. D.; Ton, N.; Rysling, S.; Quintanilla, D.; Boos, D.; Khorram, O. Therapeutic effects of in vivo administration of an inhibitor of tryptophan 2,3-dioxygenase (680c91) for the treatment of fibroids: a preclinical study. Fertil Steril 2024, 121, (4), 669-678.
29. Klavert, J.; van der Eerden, B. C. J. Fibronectin in Fracture Healing: Biological Mechanisms and Regenerative Avenues. Front Bioeng Biotechnol 2021, 9, 663357.
30. Deguine, J.; Barton, G. M. MyD88: a central player in innate immune signaling. F1000Prime Rep 2014, 6, 97.
31. Borahay, M. A.; Asoglu, M. R.; Mas, A.; Adam, S.; Kilic, G. S.; AI-Hendy, A. Estrogen Receptors and Signaling in Fibroids: Role in Pathobiology and Therapeutic Implications. Reprod Sci 2017, 24, (9), 1235-1244.
32. Kim, J. J.; Sefton, E. C. The role of progesterone signaling in the pathogenesis of uterine leiomyoma. Mol Cell Endocrinol 2012, 358, (2), 223-31.
33. DiMauro, A.; Seger, C.; Minor, B.; Amitrano, A. M.; Okeke, I.; Taya, M.; Rackow, A. R.; Kumar, D.; Kottman, R. M.; Bhagavath, B.; Hammes, S. R. Prolactin is Expressed in Uterine Leiomyomas and Promotes Signaling and Fibrosis in Myometrial Cells. Reprod Sci 2021.
34. Nowak, R. A.; Mora, S.; Diehl, T.; Rhoades, A. R.; Stewart, E. A. Prolactin is an autocrine or paracrine growth factor for human myometrial and leiomyoma cells.
Gynecol Obstet Invest 1999, 48, (2), 127-32. 35. Cetin, E.; AI-Hendy, A.; Ciebiera, M. Non-hormonal mediators of uterine fibroid growth. Curr Opin Obstet Gynecol 2020, 32, (5), 361-370.
36. Gentry, C. C.; Okolo, S. O.; Fong, L. F.; Crow, J. C.; Maclean, A. B.; Perrett, C. W. Quantification of vascular endothelial growth factor-A in leiomyomas and adjacent myometrium. Clin Sci (Lond) 2001, 101, (6), 691-5.
37. Liang, M.; Wang, H.; Zhang, Y.; Lu, S.; Wang, Z. Expression and functional analysis of platelet-derived growth factor in uterine leiomyomata. Cancer Biol Ther 2006, 5, (1), 28-33.
38. Dolcet, X.; Llobet, D.; Pallares, J.; Matias-Guiu, X. NF-kB in development and progression of human cancer. Virchows Arch 2005, 446, (5), 475-82.
39. Fan, Y.; Dutta, J.; Gupta, N.; Fan, G.; Gélinas, C. Regulation of programmed cell death by NF-kappaB and its role in tumorigenesis and therapy. Adv Exp Med Biol 2008, 615, 223-50.
40. Verzella, D.; Pescatore, A.; Capece, D.; Vecchiotti, D.; Ursini, M. V.; Franzoso, G.; Alesse, E.; Zazzeroni, F. Life, death, and autophagy in cancer: NF-KB turns up everywhere. Cell Death Dis 2020, 11, (3), 210.
41. Mirzaei, S.; Saghari, S.; Bassiri, F.; Raesi, R.; Zarrabi, A.; Hushmandi, K.; Sethi, G.; Tergaonkar, V. NF-KB as a regulator of cancer metastasis and therapy response: A focus on epithelial-mesenchymal transition. J Cell Physiol 2022, 237, (7), 2770-2795.
42. Yeh, D. W.; Huang, L. R.; Chen, Y. W.; Huang, C. F.; Chuang, T. H. Interplay between Inflammation and Stemness in Cancer Cells: The Role of Toll-Like Receptor Signaling. J Immunol Res 2016, 2016, 4368101.
43. Li, F.; Zhang, J.; Arfuso, F.; Chinnathambi, A.; Zayed, M. E.; Alharbi, S. A.; Kumar, A. P.; Ahn, K. S.; Sethi, G. NF-KB in cancer therapy. Arch Toxicol 2015, 89, (5), 711-31.
44. Chuang, T. D.; Khorram, O. Regulation of Cell Cycle Regulatory Proteins by MicroRNAs in Uterine Leiomyoma. Reprod Sci 2019, 26, (2), 250-258.
45. Davidson, B.; Abeler, V. M.; Hellesylt, E.; Holth, A.; Shih Ie, M.; Skeie-Jensen, T.; Chen, L.; Yang, Y.; Wang, T. L. Gene expression signatures differentiate uterine endometrial stromal sarcoma from leiomyosarcoma. Gynecol Oncol 2013, 128, (2), 349-55.
46. Wu, X.; Levine, A. J. p53 and E2F-1 cooperate to mediate apoptosis. Proc Natl Acad Sci USA 1994, 91, (9), 3602-6.
47. You, H.; Lin, H.; Zhang, Z. CKS2 in human cancers: Clinical roles and current perspectives (Review). Mol Clin Oncol 2015, 3, (3), 459-463.
48. Chen, L.; Ruan, Y.; Wang, X.; Min, L.; Shen, Z.; Sun, Y.; Qin, X. BAY 11-7082, a nuclear factor-kappaB inhibitor, induces apoptosis and S phase arrest in gastric cancer cells. J Gastroenterol 2014, 49, (5), 864-74.
49. Pham, L. V.; Tamayo, A. T.; Yoshimura, L. C.; Lo, P.; Ford, R. J. Inhibition of constitutive NF-kappa B activation in mantle cell lymphoma B cells leads to induction of cell cycle arrest and apoptosis. J Immunol 2003, 171, (1), 88-95.
50. Rauert-Wunderlich, H.; Siegmund, D.; Maier, E.; Giner, T.; Bargou, R. C.; Wajant, H.; StOhmer, T. The IKK inhibitor Bay 11-7082 induces cell death independent from inhibition of activation of NFKB transcription factors. PLoS One 2013, 8, (3), e59292.
51. Larson, C.; Oronsky, B.; Carter, C. A.; Oronsky, A.; Knox, S. J.; Sher, D.; Reid, T. R. TGF-beta: a master immune regulator. Expert Opin Ther Targets 2020, 24, (5), 427-438.
52. Meng, X. M.; Nikolic-Paterson, D. J.; Lan, H. Y. TGF-β: the master regulator of fibrosis. Nat Rev Nephrol 2016, 12, (6), 325-38.
53. Caja, L.; Dituri, F.; Mancarella, S.; Caballero-Diaz, D.; Moustakas, A.; Giannelli, G.; Fabregat, I. TGF-β and the Tissue Microenvironment: Relevance in Fibrosis and Cancer. Int J Mol Sci 2018, 19, (5).
54. Halder, S. K.; Goodwin, J. S.; AI-Hendy, A. 1,25-Dihydroxyvitamin D3 reduces TGF-beta3-induced fibrosis-related gene expression in human uterine leiomyoma cells. J Clin Endocrinol Metab 2011, 96, (4), E754-62.
55. Norian, J. M.; Malik, M.; Parker, C. Y.; Joseph, D.; Leppert, P. C.; Segars, J. H.; Catherino, W. H. Transforming growth factor beta3 regulates the versican variants in the extracellular matrix-rich uterine leiomyomas. Reprod Sci 2009, 16, (12), 1153-64.
56. Arici, A.; Sozen, I. Transforming growth factor-beta3 is expressed at high levels in leiomyoma where it stimulates fibronectin expression and cell proliferation. Fertil Steril 2000, 73, (5), 1006-11.
57. Joseph, D. S.; Malik, M.; Nurudeen, S.; Catherino, W. H. Myometrial cells undergo fibrotic transformation under the influence of transforming growth factor beta-3. Fertil Steril 2010, 93, (5), 1500-8.
58. Freudlsperger, C.; Bian, Y.; Contag Wise, S.; Burnett, J.; Coupar, J.; Yang, X.; Chen, Z.; Van Waes, C. TGF-β and NF-KB signal pathway cross-talk is mediated through TAK1 and SMAD7 in a subset of head and neck cancers. Oncogene 2013, 32, (12), 1549-59.
59. Fernandez-Gonzalez, A. TGF-β and NF-KB Cross-Talk: Unexpected Encounters in the Developing Lung. Am J Respir Cell Mol Biol 2021, 64, (3), 275-276.
60. Rameshwar, P.; Narayanan, R.; Qian, J.; Denny, T. N.; Colon, C.; Gascon, P. NF-kappa B as a central mediator in the induction of TGF-beta in monocytes from patients with idiopathic myelofibrosis: an inflammatory response beyond the realm of homeostasis. J Immunol 2000, 165, (4), 2271-7.
61. Brandl, M.; Seidler, B.; Haller, F.; Adamski, J.; Schmid, R. M.; Saur, D.; Schneider, G. IKK(alpha) controls canonical TGF(ss)-SMAD signaling to regulate genes expressing SNAIL and SLUG during EMT in panc1 cells. J Cell Sci 2010, 123, (Pt 24), 4231-9.
62. Kim, S. I.; Choi, M. E. TGF-β-activated kinase-1: New insights into the mechanism of TGF-β signaling and kidney disease. Kidney Res Clin Pract 2012, 31, (2), 94-105.
63. Vallet, S. D.; Ricard-Blum, S. Lysyl oxidases: from enzyme activity to extracellular matrix cross-links. Essays Biochem 2019, 63, (3), 349-364.
64. Wei, S.; Gao, L.; Wu, C.; Qin, F.; Yuan, J. Role of the lysyl oxidase family in organ development (Review). Exp Ther Med 2020, 20, (1), 163-172.
65. Johnston, K. A.; Lopez, K. M. Lysyl oxidase in cancer inhibition and metastasis. Cancer Lett 2018, 417, 174-181.
66. Wang, T. H.; Hsia, S. M.; Shieh, T. M. Lysyl Oxidase and the Tumor Microenvironment. Int J Mol Sci 2016, 18, (1).
67. Chen, W.; Yang, A.; Jia, J.; Popov, Y. V.; Schuppan, D.; You, H. Lysyl Oxidase (LOX) Family Members: Rationale and Their Potential as Therapeutic Targets for Liver Fibrosis. Hepatology 2020, 72, (2), 729-741.
68. Adamopoulos, C.; Piperi, C.; Gargalionis, A. N.; Dalagiorgou, G.; Spilioti, E.; Korkolopoulou, P.; Diamanti-Kandarakis, E.; Papavassiliou, A. G. Advanced glycation end products upregulate lysyl oxidase and endothelin-1 in human aortic endothelial cells via parallel activation of ERK1/2-NF-KB and JNK-AP-1 signaling pathways. Cell Mol Life Sci 2016, 73, (8), 1685-98.
69. Laczko, R.; Csiszar, K. Lysyl Oxidase (LOX): Functional Contributions to Signaling Pathways. Biomolecules 2020, 10, (8).
70. Moravek, M. B.; Bulun, S. E. Endocrinology of uterine fibroids: steroid hormones, stem cells, and genetic contribution. Curr Opin Obstet Gynecol 2015, 27, (4), 276-83.
71. Bakas, P.; Liapis, A.; Vlahopoulos, S.; Giner, M.; Logotheti, S.; Creatsas, G.; Meligova, A. K.; Alexis, M. N.; Zoumpourlis, V. Estrogen receptor alpha and beta in uterine fibroids: a basis for altered estrogen responsiveness. Fertil Steril 2008, 90, (5), 1878-85.
72. McKay, L. I.; Cidlowski, J. A. Cross-talk between nuclear factor-kappa B and the steroid hormone receptors: mechanisms of mutual antagonism. Mol Endocrinol 1998, 12, (1), 45-56.
73. Deroo, B. J.; Archer, T. K. Differential activation of the IkappaBalpha and mouse mammary tumor virus promoters by progesterone and glucocorticoid receptors. J Steroid Biochem Mol Biol 2002, 81, (4-5), 309-17.
74. King, A. E.; Collins, F.; Klonisch, T.; Sallenave, J. M.; Critchley, H. O.; Saunders, P. T. An additive interaction between the NFkappaB and estrogen receptor signalling pathways in human endometrial epithelial cells. Hum Reprod 2010, 25, (2), 510-8.
75. DiMauro, A.; Seger, C.; Minor, B.; Amitrano, A. M.; Okeke, I.; Taya, M.; Rackow, A. R.; Kumar, D.; Kottman, R. M.; Bhagavath, B.; Hammes, S. R. Prolactin is Expressed in Uterine Leiomyomas and Promotes Signaling and Fibrosis in Myometrial Cells. Reprod Sci 2022, 29, (9), 2525-2535.
76. Uimari, O.; Subramaniam, K. S.; Vollenhoven, B.; Tapmeier, T. T. Uterine Fibroids (Leiomyomata) and Heavy Menstrual Bleeding. Front Reprod Health 2022, 4, 818243.
77. Yang, Q.; AI-Hendy, A. Update on the Role and Regulatory Mechanism of Extracellular Matrix in the Pathogenesis of Uterine Fibroids. Int J Mol Sci 2023, 24, (6).
78. Shimamura, T.; Hsu, T. C.; Colburn, N. H.; Bejcek, B. E. Activation of NF-kappaB is required for PDGF-B chain to transform NIH3T3 cells. Exp Cell Res 2002, 274, (1), 157-67.
79. Gurbuz, N.; Asoglu, M. R.; Ashour, A. A.; Salama, S.; Kilic, G. S.; Ozpolat, B. A selective serotonin 5-HT1 B receptor inhibition suppresses cells proliferation and induces apoptosis in human uterine leiomyoma cells. Eur J Obstet Gynecol Reprod Biol 2016, 206, 114-119.
80. Barnabei, L.; Laplantine, E.; Mbongo, W.; Rieux-Laucat, F.; Weil, R. NF-KB: At the Borders of Autoimmunity and Inflammation. Front Immunol 2021, 12, 716469.
81. Dumin, J.; Wilcox, B. D.; Otterness, I.; Melendez, J. A.; Huang, C.; Jeffrey, J. J. Serotonin-mediated production of interstitial collagenase by uterine smooth muscle cells requires interleukin-1 alpha, but not interleukin-1 beta. J Biol Chem 1998, 273, (39), 25488-94.
82. Kawai, T.; Akira, S. Toll-like receptors and their crosstalk with other innate receptors in infection and immunity. Immunity 2011, 34, (5), 637-50.
83. Lim, K. H.; Staudt, L. M. Toll-like receptor signaling. Cold Spring Harb Perspect Biol 2013, 5, (1), a011247.
84. Yu, H.; Lin, L.; Zhang, Z.; Zhang, H.; Hu, H. Targeting NF-KB pathway for the therapy of diseases: mechanism and clinical study. Signal Transduct Target Ther 2020, 5,(1),209.
85. Guo, J.; Zheng, L.; Chen, L.; Luo, N.; Yang, W.; Qu, X.; Liu, M.; Cheng, Z. Lipopolysaccharide activated TLR4/NF-kappaB signaling pathway of fibroblasts from uterine fibroids. Int J Clin Exp Pathol 2015, 8, (9), 10014-25.
86. Jiang, S.; Sun, H. F.; Li, S.; Zhang, N.; Chen, J. S.; Liu, J. X. SPARC: a potential target for functional nanomaterials and drugs. Front Mol Biosci 2023, 10, 1235428.
87. Chuang, T. D.; Quintanilla, D.; Boos, D.; Khorram, O. Differential Expression of Super-Enhancer-Associated Long Non-coding RNAs in Uterine Leiomyomas. Reprod Sci 2022, 29, (10), 2960-2976.
88. Liu, F.; Zhao, Y.; Pei, Y.; Lian, F.; Lin, H. Role of the NF-kB signalling pathway in heterotopic ossification: biological and therapeutic significance. Cell Commun Signal 2024, 22, (1), 159.
89. John, B.; Naczki, C.; Patel, C.; Ghoneum, A.; Qasem, S.; Salih, Z.; Said, N. Regulation of the bi-directional cross-talk between ovarian cancer cells and adipocytes by SPARC. Oncogene 2019, 38, (22), 4366-4383.
90. D'Amato, N. C.; Rogers, T. J.; Gordon, M. A.; Greene, L. I.; Cochrane, D. R.; Spoelstra, N. S.; Nemkov, T. G.; D'Alessandro, A.; Hansen, K. C.; Richer, J. K. A TDO2-AhR signaling axis facilitates anoikis resistance and metastasis in triple-negative breast cancer. Cancer Res 2015, 75, (21), 4651-64.
91. Qin, Z.; Zhou, M. TDO2 deficiency attenuates the hepatic lipid deposition and liver fibrosis in mice with diet-induced non-alcoholic fatty liver disease. Heliyon 2023, 9, (11), e22464.
92. Wu, Z.; Yan, L.; Lin, J.; Ke, K.; Yang, W. Constitutive TDO2 expression promotes liver cancer progression by an autocrine IL-6 signaling pathway. Cancer Cell Int 2021, 21, (1), 538.
93. Chuang, T. D.; Rehan, A.; Khorram, O. Tranilast induces MiR-200c expression through blockade of ReIA/p65 activity in leiomyoma smooth muscle cells. Fertil Steril 2020, 113, (6), 1308-1318.
94. Wang, H.; Garzon, R.; Sun, H.; Ladner, K. J.; Singh, R.; Dahlman, J.; Cheng, A.; Hall, B. M.; Qualman, S. J.; Chandler, D. S.; Croce, C. M.; Guttridge, D. C. NF-kappaB-YY1-miR-29 regulatory circuitry in skeletal myogenesis and rhabdomyosarcoma. Cancer Cell 2008, 14, (5), 369-81.
95. Ikhena, D. E.; Liu, S.; Kujawa, S.; Esencan, E.; Coon, J. S. t.; Robins, J.; Bulun, S. E.; Yin, P. RANKL/RANK Pathway and Its Inhibitor RANK-Fc in Uterine Leiomyoma Growth. J Clin Endocrinol Metab 2018, 103, (5), 1842-1849.

### Example 2

### 1. Materials and Methods

### Reagents and Spheroid Cell Culture.

Leiomyoma smooth muscle cells (LSMC) were cultured in DMEM supplemented with 10% fetal bovine serum until reaching confluence with a change of media every 2-3 days. Cells at passages p1 to p3 were used for all experiments. Isolated LSMC were plated in 6-well plates (1.5×10⁵ cells/well) which were coated with 0.5% agarose gel and incubated 48 hours for spheroid formation with size ranging from 50 μm to 250 μm in diameter (1). For blocking IκBα phosphorylation and nuclear translocation of NFκB, LSMC spheroids were treated with 5 μM of Bay 11-7082 (Cayman Chemical, Ann Arbor, Michigan) for 24 hours. Cell culture experiments were performed at least three times using LSMC obtained from different patients. All supplies for the isolation and cell culture were purchased from Sigma-Aldrich (St. Louis, MO), Invitrogen (Carlsbad, CA) and Fisher Scientific (Atlanta, GA).

### Fibroid Specimens Collection.

Portions of uterine fibroids (intramural, 2-5 cm in diameters) (n=13) were obtained from patients at Harbor-UCLA Medical Center. Prior approval from the Institutional Review Board (18CR-31752-01 R) at the Lundquist Institute was obtained. Informed consent was obtained from all the patients participating in the study who were not taking any hormonal medications for at least 3 months prior to surgery. The fibroids used in this study were from African Americans (n=3), Hispanics (n=9) and Asians (n=1) women aged 35-57 years (mean 46±6.5 years). The MED12 mutation status of all fibroids was determined by PCR amplification and Sanger sequencing (Laragen Inc. Culver City, CA). Seven tumors were found to have missense mutations in MED12 exon 2. The missense mutations in exon 2 included c.130G>A (p.Gly44Ser) (n=4), c.131 G>A (p.Gly44Asp) (n=2) and c.131 G>T (p.Gly44Val) (n=1). The tissues were snap frozen and stored in liquid nitrogen for further analysis as previously described (2, 3).

### Fibroid Animal Model.

The protocol (31162-02) was approved by the IACUC at the Lundquist Institute at Harbor-UCLA Medical center. 9-12-weeks-old female ovariectomized SCID/Beige mice (Charles River Laboratories) were implanted with pellets (Innovative Research of America) containing estradiol (0.05 mg/90 d release) and progesterone (25 mg/60 d release) as previously reported (4, 5). Freshly obtained fibroid was cut aseptically into small pieces (4-5-mm³ cubes) using a razor blade and weighed. Equal weights of the explants from the same patient were implanted in the flank of mice which received Bay 11-7082 or vehicle thus allowing comparison of each treated tumor to its own control. After 3 days of recovery mice were injected daily intraperitoneal (i.p.) with vehicle (1% DMSO) or Bay 11-7082 (5 mg/kg/every other day). The dose of Bay 11-7082 used was based on a prior publication examining its efficacy in a mouse model of gastric cancer (6). After two months of treatment animals were sacrificed, and blood was obtained by cardiac puncture. Portions of liver, kidney, heart were dissected and frozen. Tumor explants were carefully dissected free of surrounding tissue, weighed and frozen. Animals were weighed before and after treatment with Bay 11-7082 or vehicle.

### Histology.

After fixation with 4% paraformaldehyde and routine dehydration, a portion of xenograft samples were embedded in paraffin. To evaluate histologic characteristics, 5-μm-thick paraffin sections of xenografts were stained with hematoxylin and eosin (H-3502, Vector Laboratories, Inc.) and Masson's trichrome stain (HT15-1 KT, Sigma-Aldrich) according to the manufacturer's instructions.

### Safety Test.

In order to determine if Bay 11-7082 administration was safe, the plasma collected from the mice was tested after 8 weeks of treatment with vehicle or Bay 11-7082. The plasma (100 μL) was pipetted into the Comprehensive Diagnostic Profile Rotor (#500-1038, Abaxis, Union City, CA, USA) and the values of glucose, creatinine, BUN, phosphorus, sodium, albumin, alkaline phosphatase, serum glutamic pyruvic transaminase (SGPT; ALT), total protein, globulin, total bilirubin and amylase were analyzed using VetScan VS2 chemistry analyzer (Abaxis, Union City, CA, USA).

### Quantitative RT-PCR.

Total RNA was extracted from the xenografts using TRIzol (Thermo Fisher Scientific Inc., Waltham, MA). RNA concentration and integrity was determined using a Nanodrop 2000c spectrophotometer (Thermo Scientific, Wilmington, DE) and Agilent 2100 Bioanalyzer (Agilent Technologies, Santa Clara, CA) as previously described (7, 8). Briefly, 2 μg of RNA was reverse transcribed using random primers for selected genes according to the manufacturer's guidelines (Applied Biosystems, Carlsbad, CA). Quantitative RT-PCR was carried out using SYBR gene expression master mix (Applied Biosystems). Reactions were incubated for 10 min at 95° C. followed by 40 cycles for 15 seconds at 95° C. and 1 min at 60° C. The expression levels of selected genes were quantified using Invitrogen StepOne System with FBXW2 (F-box and WD repeat domain containing 2) used for normalization (9). All reactions were run in triplicate and relative mRNA expression was determined using the comparative cycle threshold method (2−ΔΔCq), as recommended by the supplier (Applied Biosystems). Values were expressed as fold change compared to the control group. The primer sequences used were as follows: IL8 (sense, 5′-CTTGGCAGCCTTCCTGATTT-3′(SEQ ID NO:49); antisense, 5′-TTCTTTAGCACTCCTTGGCAAAA-3′(SEQ ID NO:50)); COL1Al (sense, 5′-CCAATGGTGCTCCTGGTATT-3′(SEQ ID NO:51); antisense, 5′-GTTCACCGCTGTTACCCTT-3′(SEQ ID NO:52)); COL3A1 (sense, 5′-ATTATTTTGGCACAACAGGAAGCT-3′(SEQ ID NO:53); antisense, 5′-TCCGCATAGGACTGACCAAGAT-3′(SEQ ID NO:54)); FN1 (sense, 5′-ACCGAAATCACAGCCAGTAG-3′(SEQ ID NO:55); antisense, 5′-CCTCCTCACTCAGCTCATATTC-3′(SEQ ID NO:56)); CCND1 (sense, 5′-GCCCTCTGTGCCACAGATGT-3′(SEQ ID NO:57); antisense, 5′-CCCCGCTGCCACCAT-3′(SEQ ID NO:58)); E2F1 (sense, 5′-GGACTCTTCGGAGAACTTTCAGATC-3′(SEQ ID NO:59); antisense, 5′-GGGCACAGGAAAACATCGA-3′(SEQ ID NO:60)); TGF-β3 (sense, 5′-CGGGCTTTGGACACCAATTA-3′(SEQ ID NO:61); antisense, 5′-GGGCGCACACAGCAGTTC-3′(SEQ ID NO:62)); DNMT1 (sense, 5′-GAACCAACGGAGAAAAAAATGG-3′(SEQ ID NO:63); antisense, 5′-GGGAGGGTGGGTCTTGGA-3′(SEQ ID NO:64)); DNMT3A (sense, 5′-GGTTCGGAGACGGCAAATT-3′(SEQ ID NO:65); antisense, 5′-GGAACGCACTGCAAAACGA-3′(SEQ ID NO:66)); MyD88 (sense, 5′-CCTGGCTGCTCTCAACAT-3′(SEQ ID NO:67); antisense, 5′-CGGATCTCCAAGTACTCAAAGT-3′(SEQ ID NO:68)); CDK2 (sense, 5′-TTCCCCTCATCAAGAGCTATCTGT-3′(SEQ ID NO:69); antisense, 5′-ACCCGATGAGAATGGCAGAA-3′(SEQ ID NO:70)); EZH2 (sense, 5′-GGAGGATCACCGAGATGATAAAG-3′(SEQ ID NO:71); antisense, 5′-TTCTGCTGTGCCCTTATCTG-3′(SEQ ID NO:72)); SPARC (sense, 5′-TACATCGGGCCTTGCAAATA-3′(SEQ ID NO:73); antisense, 5′-TGTCCTCATCCCTCTCATACA-3′(SEQ ID NO:74)) and FBXW2 (sense, 5′-CCTCGTCTCTAAACAGTGGAATAA-3′(SEQ ID NO:75); antisense, 5′-GCGTCCTGAACAGAATCATCTA-3′(SEQ ID NO:76)).

### Immunohistochemistry.

For Immunohistochemistry, the xenografts were fixed with 4% paraformaldehyde in PBS, and subsequently transferred to PBS containing 30% sucrose (wt/vol) until equilibrated in cold (4° C.). After fixation, 5-μm-thick paraffin sections were treated three times with Histo-Clear™ (National Diagnostics, Atlanta, GA) for 5 min, and rehydrated by a sequential ethanol wash, and then incubated in target retrieval solution (Dako, Carpinteria, CA) in a microwave for 10 min in order to retrieve the antigens. For blocking, tissues were incubated for 10 minutes with 3% solution of H2O2 followed by incubation with PBS-5% normal goat serum-0.2% Triton X-100. Tissue sections were incubated with primary antibody rabbit anti-Ki67 (dilution 1:250, 27309-1-AP, Proteintech Group, Inc), rabbit anti-cleaved caspase-3 (dilution 1:50, #9664, Cell Signaling Technology), rabbit anti-CCND1 (dilution 1:50, #55506, Cell Signaling Technology) and mouse anti-E2F1 (dilution 1:50, sc-251, Santa Cruz Biotechnology, Inc.) overnight at 4° C. in a humidified chamber. The antigens were then visualized using biotinylated antibodies and streptavidin, conjugated with horseradish peroxidase. Control sections were incubated with the secondary antibody, with replacement of primary antibody with the dilution reagent (Dako). Diaminobenzidine (Dako) which served as the substrate. All the sections were counterstained with hematoxylin and eosin. Immunostained sections were examined under a microscope (Olympus IX83; Olympus Surgical Technologies America) and five representative images of each slide were quantitatively analyzed with the use of Halo software (Indica Labs Inc.) and were averaged for comparative analysis between vehicle and tranilast treatment groups in a blinded fashion.

### Statistics.

Throughout the text, results are presented as mean±SEM and analyzed by PRISM software (Graph-Pad, San Diego, CA). Dataset normality was determined by the Kolmogorov-Smirnoff test. The data presented in this study was not normally distributed and therefore non-parametric tests were used for data analysis. Comparisons involving two groups was analyzed using the Wilcoxon matched pairs signed rank test as appropriate. Statistical significance was established at P<0.05.

### II. Results

### In Vitro Data.

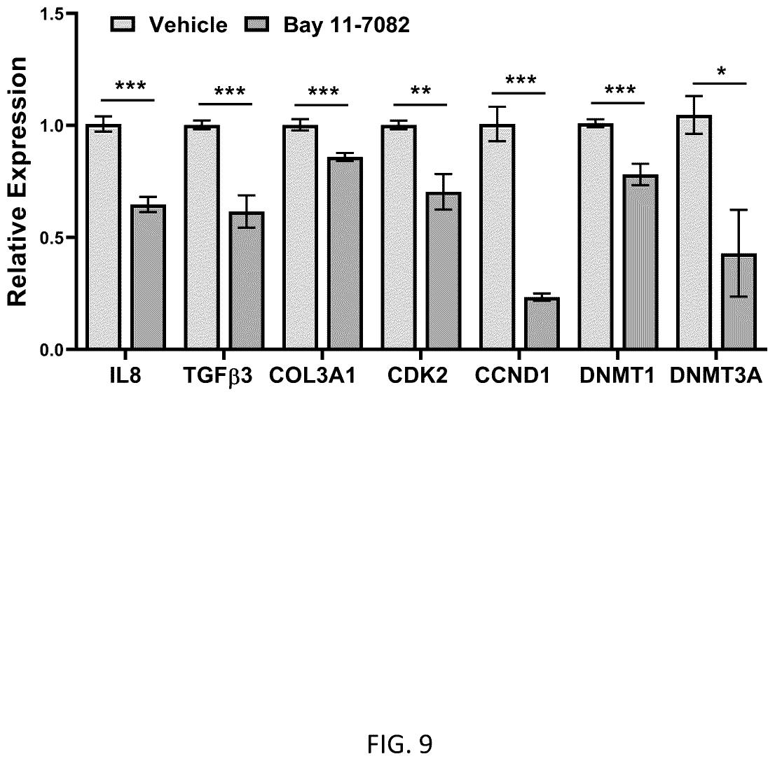

Using 3D cultured primary leiomyoma smooth muscle cells (LSMC), treatment with Bay 11-7082, a selective inhibitor NF-KB signaling (cytokine-induced IκBα phosphorylation), resulted in a significant decrease in the expression of IL8, TGFB3, Col3A1, CDK2, CCND1, DNMT1 and DNMT3A (FIG. 9), indicating that the expression of these genes which are essential to the pathogenesis of fibroids is regulated via NF-kB signaling.

### In Vivo Data.

A xenograft mouse fibroid model was used to test the efficacy of Bay 11-7082 on tumor weight, growth, and extracellular matrix deposition. Briefly, freshly obtained fibroids from hysterectomies was cut aseptically into small pieces (4-5-mm3 cubes) using a razor blade and weighed. Equal weights of the explants from the same patient were implanted in the flank of SCID mice. Mice were treated with Bay 11-7082 or vehicle thus allowing comparison of each treated tumor to its own control. After 3 days of recovery mice were injected daily intraperitoneal (i.p.) with vehicle (DMSO) or Bay 11-7082 (5 mg/kg/three times per week). The dose of Bay 11-7082 used was based on a prior publication examining its efficacy in a mouse model of gastric cancer (PMID: 23846545). After two months of treatment animals were sacrificed, and blood was obtained by cardiac puncture. Portions of liver, kidney, heart were dissected and frozen. Tumor explants were carefully dissected free of surrounding tissue, weighed and frozen. Animals were weighed before and after treatment with Bay 11-7082 or vehicle.

These results showed that treatment with Bay 11-7082 (5 mg/kg/three times per week) was well tolerated by mice with no significant effects of Bay 11-7082 on body weight (vehicle vs Bay 11-7082: 26.16g±0.49 vs 25.42g±0.89).

TABLE 1
Chemistry panel results indicate no adverse effects of in vivo Bay 11-7082
treatment

| | Vehicle | Bay 11-7082 | |
|---|---|---|---|
| Chemistry Panel Marker | (mean ± SEM) | (mean ± SEM) | P-value |
| General metabolism | | | |
| Glucose (mg/dl) | 230.9 ± 17.73 | 261.0 ± 14.63 | 0.0689 |
| Kidney function | | | |
| BUN (mg/dl) | 24.3 ± 2.8 | 27.1 ± 5.75 | 0.6484 |
| Creatinine (mg/dl) | 0.2 ± 0.29 | 0.4 ± 0.03 | 0.9394 |
| Electrolytes | | | |
| Sodium (mEq/l) | 154.4 ± 0.98 | 155.3 ± 1.35 | 0.6278 |
| Phosphorus (mg/dl) | 8.7 ± 0.77 | 10.1 ± 0.53 | 0.9883 |
| Liver function | | | |
| Alkaline phosphatase (U/l) | 105 ± 16.95 | 91.9 ± 20.05 | 0.3547 |
| Albumin (g/dl) | 3.9 ± 0.12 | 3.5 ± 0.14 | 0.0625 |
| SGPT (ALT) (U/l) | 44 ± 10.3 | 54.5 ± 8.71 | 0.5038 |
| Total protein (g/dl) | 5.2 ± 0.61 | 4.6 ± 0.16 | 0.326 |
| Globulin (g/dl) | 1.3 ± 0.5 | 1.1 ± 0.24 | 0.7863 |
| Total bilirubin (mg/dl) | 0.2 ± 0.02 | 0.3 ± 0.04 | 0.7318 |
| Pancreas function | | | |
| Amylase (U/l) | 703.9 ± 32.93 | 643.0 ± 36.82 | 0.0738 |

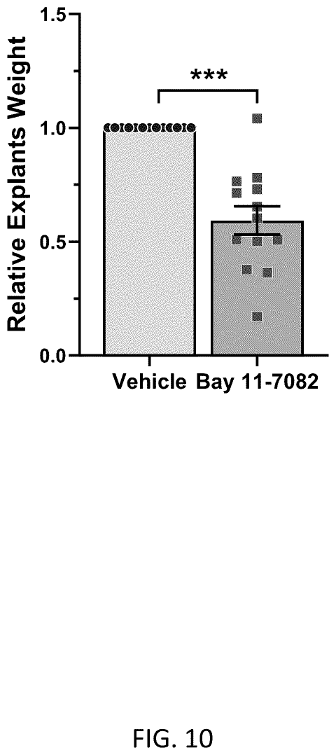

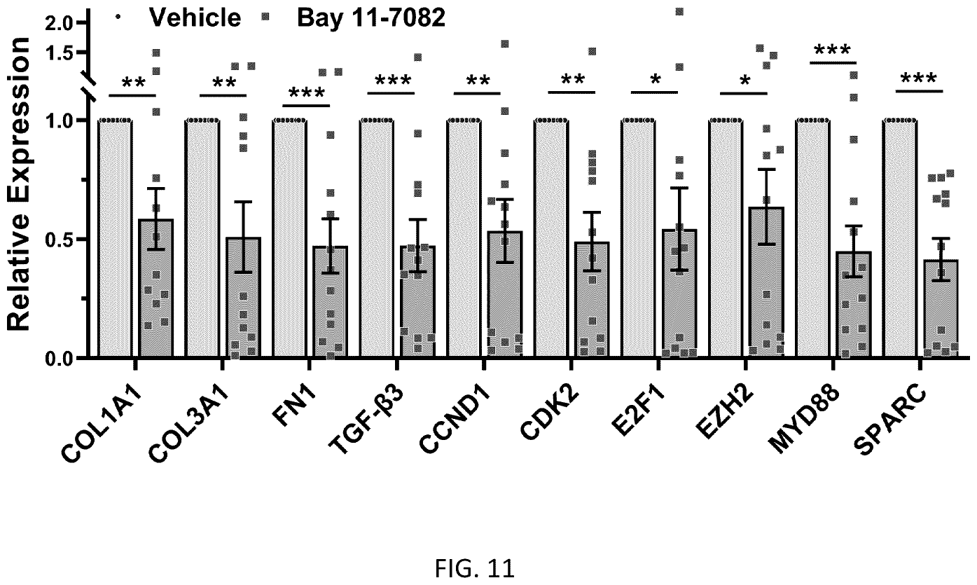

As shown in Table 1 blood plasma chemistry panel revealed no adverse effects of in vivo Bay 11-7082 treatment on carbohydrate metabolism (glucose), kidney function [blood urea nitrogen (BUN), creatinine], electrolytes (sodium, phosphorus) or liver function [alkaline phosphatase, hepatocyte glutamic pyruvic transaminase (SGPT; ALT), albumin, total protein, globulin, total bilirubin] and pancreatic amylase. Xenografts from the tranilast treated group showed a 40% reduction in tumor weight as compared to their matched controls after two months of treatment with Bay 11-7082 (FIG. 10). The expression of a number of key genes that play a key role in fibroid pathogenesis were examined by qRT-PCR, including three major components of the ECM namely COL1A1, COL3A1 and FN1, three key regulators of the cell cycle (CCND1, CDK2 and E2F1), one epigenetic modulator EZH2, two inflammatory regulator (MyD88 and SPARC), and TGF-β3 a critical pro-fibrotic growth factor known to be over expressed in fibroids. As demonstrated in FIG. 11, there was a significant decrease in the mRNA expression of COL1A1, COL3A1, FN1, TGF-β3, CCND1, CDK2, E2F1, EZH2, MyD88 and SPARC in the xenografts of Bay 11-7082 treated mice.

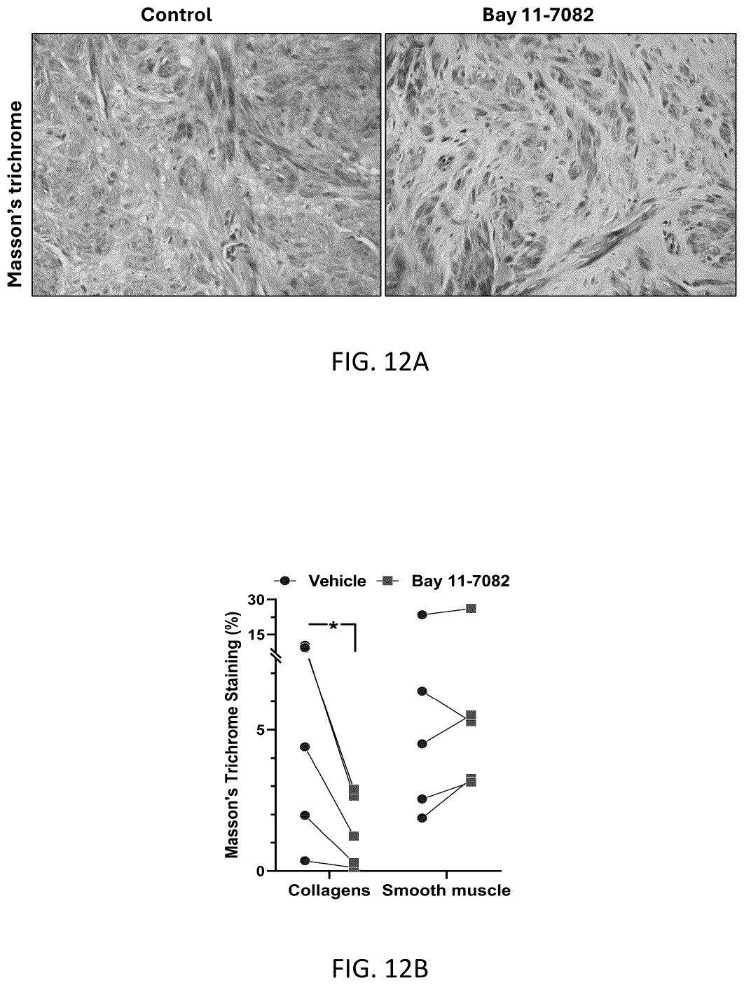

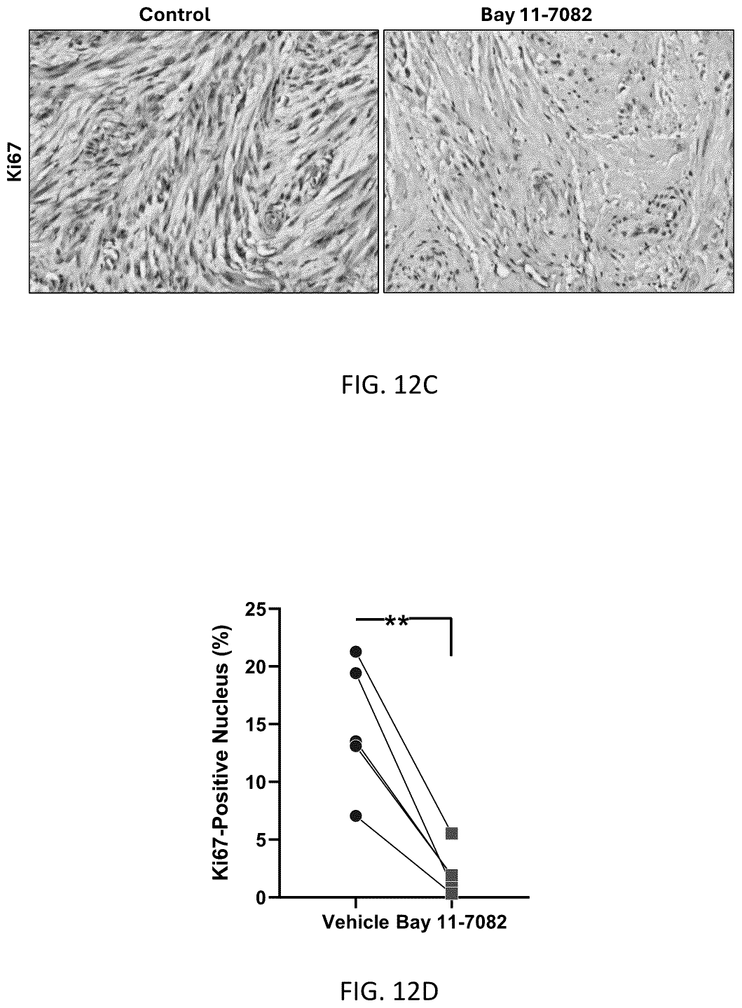

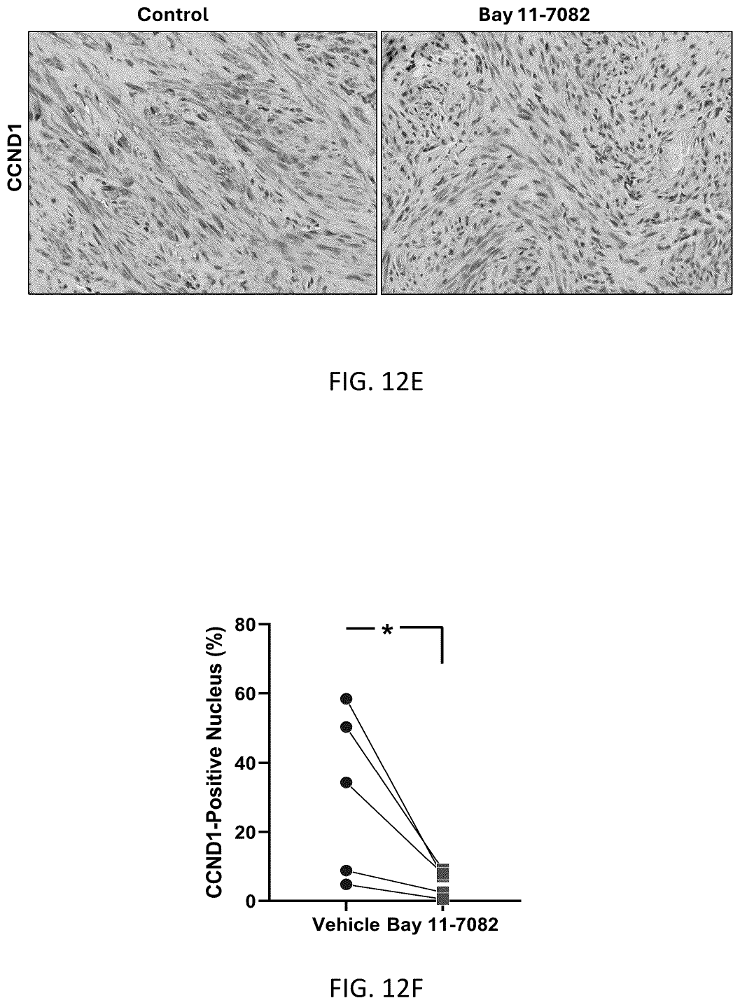

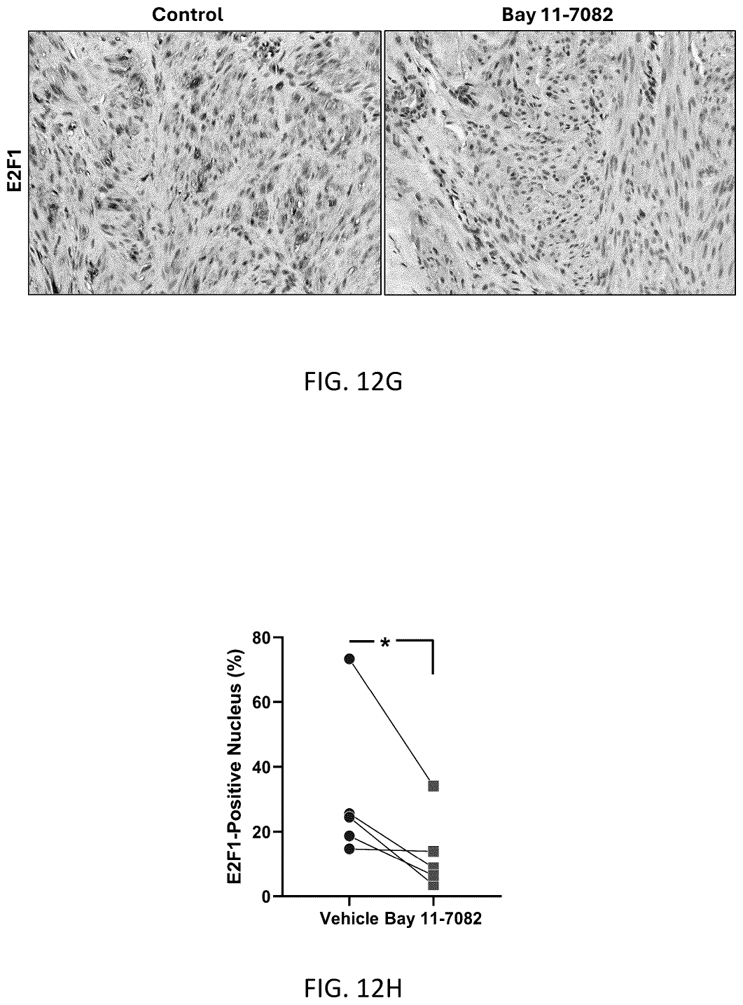

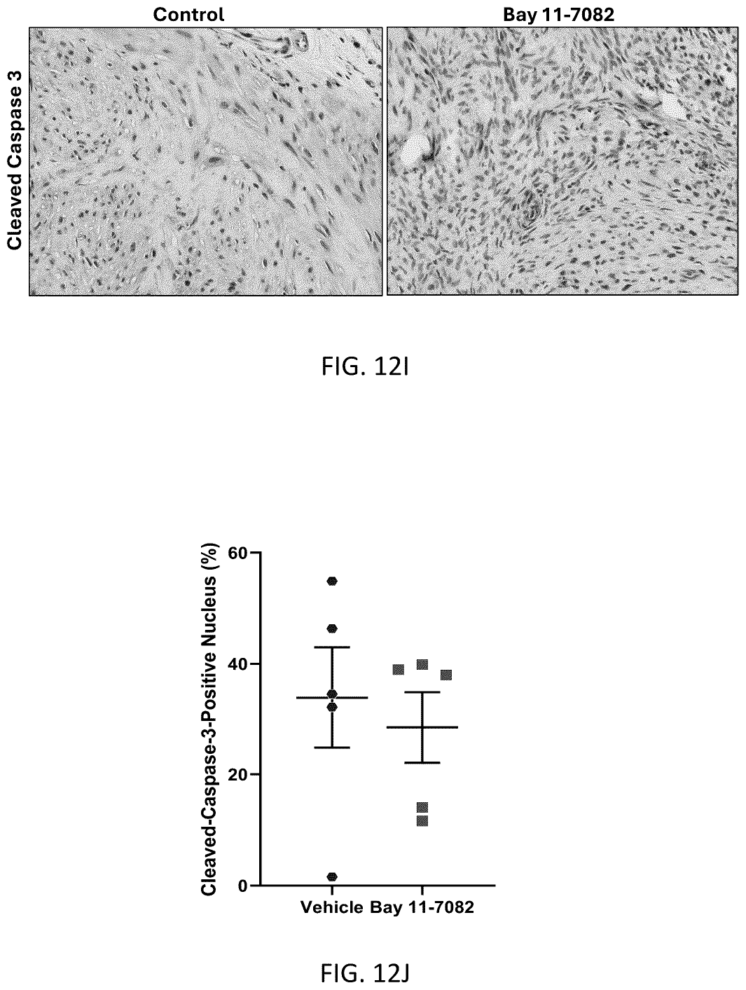

The xenografts were also subjected to histologic and immunohistochemical analysis. Masson's trichrome staining was used to assess the expression of collagen (blue stain) and smooth muscle cells (red stain) in the xenografts. As shown in FIG. 12A and FIG. 12B, there was a significant decrease in the staining for collagen (blue stain) in xenografts of the Bay 11-7082 treated mice. In addition, xenografts of Bay 11-7082 treated mice exhibited reduced cell proliferation as indicated by reduced number of nuclei staining for Ki67, CCND1 and E2F1 (FIGS. 12C-12H), whereas these xenografts exhibited no significant change in nuclei staining of cleaved caspase 3 indicative of no effect on apoptosis (FIG. 121 and FIG. 12J).

In summary, these results indicate that the nfkb inhibitor Bay 11-7082 has anti-proliferative, anti-fibrotic and anti-inflammatory drug and by virtue of these effects shrinks fibroid tumors. This preclinical study indicates that Bay-11-7802 and other nfkb inhibitors could be used for treatment of fibroids in humans.

### III. References

1. Chuang T D, Rehan A, Khorram O. Functional role of the long noncoding RNA X-inactive specific transcript in leiomyoma pathogenesis. Fertility and sterility. 2021; 115(1):238-47.
2. Chuang T D, Luo X, Panda H, Chegini N. miR-93/106b and their host gene, MCM7, are differentially expressed in leiomyomas and functionally target F3 and IL-8. Molecular endocrinology (Baltimore, Md). 2012; 26(6):1028-42.
3. Chuang T D, Panda H, Luo X, Chegini N. miR-200c is aberrantly expressed in leiomyomas in an ethnic-dependent manner and targets ZEBs, VEGFA, TIMP2, and FBLN5. Endocrine-related cancer. 2012; 19(4):541-56.
4. Ishikawa H, Ishi K, Serna V A, Kakazu R, Bulun S E, Kurita T. Progesterone is essential for maintenance and growth of uterine leiomyoma. Endocrinology. 2010; 151(6):2433-42.
5. Ono M, Yin P, Navarro A, Moravek M B, Coon J S t, Druschitz S A, et al. Paracrine activation of WNT/beta-catenin pathway in uterine leiomyoma stem cells promotes tumor growth. Proceedings of the National Academy of Sciences of the United States of America. 2013; 110(42):17053-8.
6. Chen L, Ruan Y, Wang X, Min L, Shen Z, Sun Y, et al. BAY 11-7082, a nuclear factor-kappaB inhibitor, induces apoptosis and S phase arrest in gastric cancer cells. Journal of gastroenterology. 2014; 49(5):864-74.
7. Chuang T-D, Khorram O. Cross-talk between miR-29c and transforming growth factor-β3 is mediated by an epigenetic mechanism in leiomyoma. Fertility and sterility. 2019; 112(6):1180-9.
8. Chuang T D, Quintanilla D, Boos D, Khorram O. Long Noncoding RNA MIAT Modulates the Extracellular Matrix Deposition in Leiomyomas by Sponging MiR-29 Family. Endocrinology. 2021; 162(11):bqab186.
9. Almeida T A, Quispe-Ricalde A, Montes de Oca F, Foronda P, Hernandez M M. A high-throughput open-array qPCR gene panel to identify housekeeping genes suitable for myometrium and leiomyoma expression analysis. Gynecologic oncology. 2014; 134(1):138-43.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of treating fibroid in a patient in need thereof, comprising administering to the patient an effective amount of a nuclear factor-kappa B (NF-kB) inhibitor.

2. The method according to claim 1, wherein the NF-kB inhibitor is Bay 11-7082.

3. The method according to claim 1, wherein the fibroid is a uterine fibroid.

4. The method according to claim 1, wherein the uterine fibroid is selected from the group consisting of intramural fibroid, subserosal fibroid, subserosal tumor, or submucosal fibroid.

5. The method according to claim 4, which ameliorates at least a symptom selected from the group consisting of heavy bleeding between or during periods that includes blood clots, pain in the pelvis or lower back, increased menstrual cramping, increased urination, pain during intercourse, menstruation that lasts longer than usual, pressure or fullness in your lower abdomen, and swelling or enlargement of the abdomen.

6. The method according to claim 1, wherein the subject is a mammal.

7. The method according to claim 6, wherein the mammal is a human.

8. A pharmaceutical for use in treating fibroid in a patient in need thereof, the composition comprising an effective amount of a nuclear factor-kappa B (NF-kB) inhibitor.

9. The composition according to claim 8, wherein the NF-kB inhibitor is Bay 11-7082.
